(19) United States
(12) Patent Application Publication
(10) Pub. No.: US 2023/0158078 A1
(43) Pub. Date: May 25, 2023

(54) METHODS FOR TREATING ACUTE RESPIRATORY INFLAMMATORY CONDITIONS AND CYTOKINE STORM SYNDROME

(71) Applicant: REMEDY CELL LTD., Jerusalem (IL)

(72) Inventors: Yuval SHAKED, Binyamina (IL); Michael TIMANER, Haifa (IL)

(21) Appl. No.: 18/094,501

(22) Filed: Jan. 9, 2023

Related U.S. Application Data
(63) Continuation of application No. PCT/IL2021/050841, filed on Jul. 8, 2021.
(60) Provisional application No. 63/049,663, filed on Jul. 9, 2020.

(51) Int. Cl. A61K 35/28; A61P 11/00; A61P 29/00; A61K 31/337; A61K 33/243; A61K 31/7068; A61P 31/14; A61K 45/06; C12N 5/0775

(52) U.S. Cl. CPC A61K 35/28; A61P 11/00; A61P 29/00; A61K 31/337; A61K 33/243; A61K 31/7068; A61P 31/14; A61K 45/06; C12N 5/0663; C12N 2501/06

(57) ABSTRACT

Methods of treating, ameliorating or preventing an acute respiratory disease or condition or cytokine storm in a subject in need thereof, comprising administering to the subject a pharmaceutical composition comprising an effective amount of MSCs contacted with chemotherapy or conditioned media from the MSCs contacted with chemotherapy are provided.

### CROSS REFERENCE TO RELATED APPLICATIONS

This application is a ByPass Continuation of PCT Patent Application No. PCT/IL2021/050841 having International filing date of Jul. 8, 2021, which claims the benefit of priority of U.S. Provisional Patent Application No. 63/049,663 having filing date of Jul. 9, 2020, entitled “ANTI CANCER THERAPY—ACTIVATED CELL THERAPY FOR TREATING ACUTE RESPIRATORY INFLAMMATORY CONDITIONS AND CYTOKINE STORM SYNDROME”, the contents of which are all incorporated herein by reference in its entirety.

### FIELD OF INVENTION

The present invention is in the field of cytokine storm and inflammation treatment.

### BACKGROUND OF THE INVENTION

Lung inflammatory diseases remain a leading cause for hospitalization in intensive care units and are associated with high mortality rates. Respiratory failure is often caused by acute pulmonary distress syndrome (ARDS) which is defined by noncardiogenic pulmonary oedema, acute hypoxaemia and the need for mechanical ventilation. ARDS mainly develops from other diseases, including severe lung trauma, viral or bacterial pneumonia, sepsis, aspiration of gastrointestinal contents with consequent pulmonary infection, and acute pancreatitis. Although, improved intensive care in the past 60 years has increased patient survival, the death rate remains 30-40%. Furthermore, in many patients, the combination of aggressive immune system activation caused by viral or bacterial infection, together with insufficient anti-inflammatory response leads to cytokine storm syndrome (CSS). Respiratory epithelium damage with consequent overactivation of macrophages and lymphocytes induce massive production of proinflammatory cytokines including interferons, tumor necrosis factors, interleukins, and chemokines. However, the initiated regenerative processes are limited and insufficient. Therefore, this uncontrolled inflammation causes massive local alveolar damage, as well as induces pulmonary capillary injury leading to increased systemic levels of inflammatory cytokines which can promote multi-organ dysfunction.

Recently, the outbreak of corona virus disease 2019 (COVID-19), first identified in December 2019 in Hubei province in China, has spread worldwide and became a pandemic. The disease is induced by infection with severe acute respiratory syndrome corona virus 2 (SARS-CoV-2). Among the severe symptoms of the disease are fever, dry cough, and dyspnea. Some patients having these symptoms will progress to severe pneumonia that leads to ARDS, CSS and multi-organ failure. While the average worldwide death rate from COVID-19 is 4-7%, in high-risk patients, including elderly people and patients with certain comorbidities (e.g., diabetes, autoimmune diseases, hypertension), the death rate reaches 15%. COVID-19 is characterized by a prolonged incubation period which is generally asymptomatic and varies from 2 to 14 days until the appearance of the first symptoms. Even then, the majority of patients infected with the virus develop only mild disease or even remain asymptomatic. Currently, the treatment of COVID-19 is maintained in three main directions: anti-viral therapy to treat the infection, including recently approved Remdesivir (Gilead Sciences, Inc.); anti-cytokines therapy to treat CSS, including anti-IL-6 and anti-IL-1 therapies; and finally intensive supportive care with mechanical ventilation to patients with ARDS. Still, research on physiological mechanisms and the pathology of the disease is required to choose the type and timing of the most suitable therapies, thus improving the survival of patients and to effectively managing the pandemic.

A growing body of evidence suggests that a subgroup of patients with severe COVID-19 develop life-threatening CSS. In particular, a cytokine profile resembling secondary hemophagocytic lymphohistiocytosis (sHLH), a hyperinflammatory syndrome characterized by a fulminant and fatal hypercytokinaemia with multi-organ failure is demonstrated in COVID-19 patients. sHLH is usually induced by viral infections and occurs in about 4% of sepsis cases. sHLH associated with COVID-19 disease severity is characterized by increased interleukin (IL)-2, IL-7, granulocyte-colony stimulating factor (GCSF), and other pro-inflammatory cytokines. In addition, it was shown that predictors of death of COVID-19 patients include elevated levels of ferritin and IL-6 in the blood, further suggesting that mortality might be due to virus-activated hyperinflammation. Therefore, effective COVID-19 treatment should contain effective anti-inflammatory agents, which suppress overactivation of the immune system.

Mesenchymal stem cells (MSCs) are multipotent stem cells residing in the bone marrow and mesenchymal tissues. MSCs obtain strong anti-inflammatory properties by secreting factors including PGE2, TSG6, TGFβ, and IL-10 therefore inducing vast immunosuppressive activity. Moreover, it has been previously demonstrated that MSCs possess great regeneration abilities, they effectively home to damaged tissues, reduce inflammation and immune cell influx, improve vascularization and stimulate tissue repair. In addition, activation of MSCs by chemotherapy agents further enhances the regenerative potential of MSC. There is a need for a safe and effective medicament or therapy to prevent or reduce COVID-19 infection as well as ARDS and CCS.

### SUMMARY OF THE INVENTION

The present invention provides methods of treating, ameliorating or preventing an acute respiratory disease or condition or cytokine storm in a subject in need thereof, comprising administering to the subject a pharmaceutical comprising an effective amount of MSCs contacted with paclitaxel or conditioned media from the MSCs contacted with paclitaxel.

According to a first aspect, there is provided a method of treating, ameliorating or preventing an acute respiratory disease or condition in a subject in need thereof, the method comprising administering to the subject a pharmaceutical composition comprising an effective amount of mesenchymal stem cells (MSCs) contacted with a chemotherapy or conditioned medium (CM) derived from the MSCs contacted with the chemotherapy, thereby treating, ameliorating or preventing an acute respiratory disease or condition in a subject.

According to a first aspect, there is provided a method of treating, ameliorating or preventing a cytokine storm in a subject in need thereof, the method comprising administering to the subject a pharmaceutical composition comprising an effective amount of MSCs contacted with a chemotherapy or conditioned medium (CM) derived from the MSCs contacted with the chemotherapy, thereby treating, ameliorating or preventing a cytokine storm in a subject.

According to some embodiments, the acute respiratory disease or condition is acute respiratory distress syndrome (ARDS).

According to some embodiments, the acute respiratory disease or condition is selected from severe lung trauma, sepsis, pulmonary infection, and acute pancreatitis.

According to some embodiments, the subject suffers from a pulmonary infection.

According to some embodiments, the pulmonary infection is selected from a bacterial infection, a viral infection and an infection consequent to aspiration of gastrointestinal contents.

According to some embodiments, the viral infection is a corona virus infection.

According to some embodiments, the corona virus is selected from, human coronavirus (HcoV)-NL63, HCoV-OC43, HCoV-229E, HCoV-HKUI, severe acute respiratory syndrome coronavirus (SARS-CoV-1), middle east respiratory syndrome coronavirus (MERS-CoV) and SARS-CoV-2.

According to some embodiments, the acute respiratory disease or condition is an acute pulmonary infection, and the method is a method for treating an acute pulmonary infection in a subject in need thereof.

According to some embodiments, the acute respiratory disease or condition comprises at least one of: a cytokine storm, cytokine release syndrome, secondary hemophagocytic lymphohistiocytosis (sHLH), acute respiratory distress syndrome (ARDS), unremitting fever, cytopenia, or hyperferritinaemia.

According to some embodiments, the subject does not suffer from lung fibrosis.

According to some embodiments, the subject suffers from cytokine release syndrome.

According to some embodiments, the method comprises administering the conditioned media to the subject.

According to some embodiments, the method further comprises administrating to the subject an additional drug or therapy.

According to some embodiments, the additional drug or therapy is selected from: an anti-cytokine therapy, an anti-viral drug, an analgesic drug, a corticosteroid and mechanical ventilation.

According to some embodiments, the anti-viral drug is selected from remdesivir, ribavirin, oseltamivir, zanamivir and interferon-alpha 2b.

According to some embodiments, the anti-cytokine therapy is selected from anti-IL-6, anti-IL6R and anti-IL1.

According to some embodiments, the treating comprises reduction of a proinflammatory cytokine selected from IL-6, TNFα, IL-2, IL-3, G-CSF, MIP-1α, and INFγ. improvement in a respiratory parameter or a combination thereof.

According to some embodiments, the treating comprising reduction of at least on proinflammatory cytokine selected from IL-6, TNFα, IL-2, IL-3, G-CSF, MIP-1α, and INFγ in the subject.

According to some embodiments, the reduction is a reduction in the blood of the subject.

According to some embodiments, the composition is administered by oral, sublingual, subcutaneous, intramuscular, intravenous, topical, local, intratracheal, intranasal, transdermal or rectal administration.

According to some embodiments, the composition is administered by intratracheal or intravenous administration.

According to some embodiments, the chemotherapy is selected from paclitaxel, cisplatin and gemcitabine.

According to some embodiments, the chemotherapy is selected from paclitaxel and cisplatin.

According to some embodiments, the chemotherapy is paclitaxel.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The invention is herein described, by way of example only, with reference to the accompanying drawings. With specific reference now to the drawings in detail, it is stressed that the particulars shown are by way of example and for purposes of illustrative discussion of the preferred embodiments of the present invention only and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIGS. 1A-1E: Paclitaxel-activated MSC-derived therapy improves acute pulmonary inflammation. Each of FIGS. 1A-1E, in the top panel, demonstrates computed tomography (CT) assessment of pulmonary inflammation, and in bottom panel, demonstrates Hematoxylin and Eosin (H&E) staining of lungs. Ten-to-twelve-week-old C57Bl mice were intratracheally administrated (1B-E) 5 mg/kg lipopolysaccharides (LPS) or (1A) vehicle control (4 mice/group). Mice were either left untreated or intratracheally (IT) administered conditioned medium (CM) from (1C) control mesenchymal stem cells (MSCs), (1D) cisplatin-activated MSCs, or (1E) paclitaxel-activated MSCs. Treatment was given daily for five days. Before the endpoint, pulmonary inflammation was assessed by CT. Consequently, lungs were removed and processed for histopathology. Hematoxylin and Eosin (H&E) staining of lung slices is demonstrated. Representative images for each panel are shown.

FIG. 2: Paclitaxel-activated MSC-derived therapy restores respiratory rate of mice with acute pulmonary inflammation. Bar graph showing respiration rate. Ten-to-twelve-week-old C57Bl mice were intratracheally (IT) administrated 5 mg/kg lipopolysaccharides (LPS) or treated with vehicle control (4 mice/group). Mice were either left untreated or IT administered conditioned medium (CM) from control MSCs, cisplatin, or paclitaxel-activated MSCs. Treatment was given daily for 5 days. Before the endpoint, pulmonary inflammation was assessed by CT scanning and respiratory rate was recorded.

FIG. 3: Conditioned medium from paclitaxel-activated MSCs inhibits immune cell infiltration into lungs with acute inflammation. Bar graph showing immune cell number. Ten-to-twelve-week-old C57Bl mice were intratracheally (IT) administrated 5 mg/kg lipopolysaccharides (LPS) or vehicle control. Mice were either left untreated or IT administered CM from cisplatin- or paclitaxel-activated MSCs. Treatment was given daily for five days. At the endpoint, bronchoalveolar lavage fluid (BALF) was collected, and immune cells were counted. The number of cells in BALF is shown (n=4 mice/group).

FIGS. 4A-4B: Chemotherapy-activated MSC-derived therapy attenuates pulmonary inflammation. (4A) Bar graphs showing immune cell percentage. The cells from FIG. 3 were immunostained for MDSCs. The cells were then analyzed by flow cytometry, and the percentage of cells in each sample was tested. (4B) Bar graphs of immune cells that were differentiated into a macrophage state as detailed in the Materials and Methods. The bone marrow derived macrophages were cultured with conditioned medium from chemotherapy-activated MSCs, and then analyzed by flow cytometry to evaluate the state of the macrophages. In the graph, the percentage of pro-regenerative M2 macrophages is shown.

FIGS. 5A-5F: Paclitaxel-activated MSC-derived therapy caused better recovery of acute pulmonary inflammation compared to other chemo-activators. Each of FIGS. 5A-5F demonstrates computed tomography (CT) assessment of pulmonary inflammation. Ten-to-twelve-week-old C57Bl mice were intratracheally administrated (5B-F) 5 mg/kg lipopolysaccharides (LPS) or (5A) vehicle control (4 mice/group). Mice were either left untreated or intratracheally (IT) administered conditioned medium (CM) from (5C) control mesenchymal stem cells (MSCs), (5D) gemcitabine-, (5E) cisplatin-, or (5F) paclitaxel-activated MSCs. Treatment was given daily for five days. Before the endpoint, pulmonary inflammation was assessed by CT. Representative images for each panel are shown.

FIGS. 6A-6D: Intratracheal and intraperitoneal administration of paclitaxel-activated MSC-derived therapy improves acute pulmonary inflammation in mice. Ten-to-twelve-week-old C57Bl mice were intratracheally administrated (6B-D) 5 mg/kg lipopolysaccharides (LPS) or (6A) vehicle control (4 mice per group). Mice were either left untreated or (6C) intraperitoneally (IP) administered (100 μl) or (6D) intratracheally (IT) administered (70 μl) conditioned medium (CM) from paclitaxel-activated MSCs. Treatment was given daily for five days. Before the endpoint, pulmonary inflammation was assessed by CT (top panels). Consequently, lungs were removed and processed for histopathology. Hematoxylin and Eosin (H&E) staining of lungs is demonstrated (bottom panels). Representative images for each panel are shown.

FIG. 7: Intratracheal and intraperitoneal administration of paclitaxel-activated MSC-derived therapy improves respiratory rate in mice with acute pulmonary inflammation. Bar graph showing respiration rate. Ten-twelve-week-old C57Bl mice were intratracheally administrated 5 mg/kg lipopolysaccharides (LPS) or vehicle control (4 mice/group). Mice were either left untreated or administered via intraperitoneal injection (IP, 100 μl) or intratracheal instillation (IT, 70 μl) conditioned medium (CM) from paclitaxel-activated MSCs. Treatment was given daily for 5 days. Before the endpoint, respiratory rate was recorded.

FIG. 8: A volcano plot showing that paclitaxel-activated MSC-derived therapy reduces plasma levels of pro-inflammatory cytokines in mice with acute pulmonary inflammation and increases anti-inflammatory cytokines. Ten-twelve-week-old C57Bl mice were intratracheally administrated 5 mg/kg lipopolysaccharides (LPS). Mice were either left untreated or administered by intratracheal instillation (IT, 70 μl) conditioned medium (CM) from paclitaxel-activated MSCs (4 mice/group). Treatment was given daily for five days. At the endpoint, peripheral blood was collected and pooled for each group. The expression levels of various inflammatory cytokines in plasma were evaluated using proteome profiler mouse XL cytokine array (R&D systems). The data is presented as a volcano plot.

FIGS. 9A-9D: Paclitaxel-activated MSC-derived therapy outperforms anti-IL-6R blockade therapy for the treatment of acute pulmonary inflammation. Ten-twelve-week-old C57Bl mice were intratracheally administrated (9B-D) 5 mg/kg lipopolysaccharides (LPS) or (9A) vehicle control (4 mice/group). Mice were either left untreated or treated with (9C-D) anti-mouse IL-6R antibody (10 mg/kg; BioXcell) (9C) injected intraperitoneally (IP) or (9D) instilled intratracheally (IT, 70 μl) with conditioned medium (CM) from paclitaxel-activated MSCs. Treatment was given daily for five days. Before the endpoint, pulmonary inflammation was assessed by CT (top panel). Consequently, lungs were removed and processed for histopathology. Hematoxylin and Eosin (H&E) staining of lungs is demonstrated (bottom panel). Representative images for each panel are shown.

FIGS. 10A-10C: Paclitaxel-activated MSC-derived therapy better improvement in lung physiology in mice with acute pulmonary inflammation than anti-IL-6R blockade therapy. (10A-C) Bar charts showing (10A) inflammatory tissue (10B) and inhalation volumes calculated using CTAn software (Bruker Corporation) and (10C) respiration rates. Ten-twelve-week-old C57Bl mice were intratracheally administrated 5 mg/kg lipopolysaccharides (LPS) or vehicle control (4 mice/group). Mice were either left untreated or treated with anti-mouse IL-6R antibody (10 mg/kg; BioXcell) injected intraperitoneally (IP) or instilled intratracheally (IT, 70 μl) with conditioned medium (CM) from paclitaxel-activated MSCs. Treatment was given daily for five days. Before the endpoint, pulmonary function was assessed by CT.

FIGS. 11A-I: Paclitaxel-activated MSC-derived therapy is safe and non-toxic in healthy mice. Ten-twelve-week-old C57Bl mice were either left untreated or administered via intraperitoneal injection (IP, 100 μl) or intratracheal instillation (IT, 70 μl) with conditioned medium (CM) from paclitaxel-activated MSC (4 mice/group). Treatment was given daily for 8 days. In the endpoint, organs were collected, fixated and examined for pathological lesions and histopathological changes in the tissues. Representative images of the following tested organs are provided: (11A) Brain, (11B) spleen, (11C) stomach, (11D) heart, (11E) Kidney, (11F) small intestine, (11G) lungs, (11H) liver, (11I) Colon.

### DETAILED DESCRIPTION OF THE INVENTION

The present invention, in some embodiments, provides methods of treating, ameliorating or preventing an acute respiratory disease or condition in a subject in need thereof. The present invention further concerns a method of treating preventing or ameliorating a cytokine storm in a subject in need thereof.

Surprisingly, mesenchymal stem cells (MSC), macrophages, fibroblasts, and T cells, that are activated by an anticancer drug (termed here “repairing cells”) or the conditioned media thereof were found surprisingly effective in treating or managing acute diseases or conditions. In particular, it was surprisingly found that the conditioned media (CM) from MSCs contacted with paclitaxel was superior to CM from untreated MSCs, MSCs contacted with cisplatin and MSCs contacted with gemcitabine. Though it was known that MSCs activated with anticancer therapeutics could improve the ability of MSCs to treat chronic damage, such as fibrosis, it was not known that these cells and their secretome we well suited to treat acute conditions such as infection and cytokine storm. Further, it was heretofore unknown that paclitaxel activation was particularly well suited for treatment of these types of disease.

By a first aspect, there is provided a method of treating, ameliorating or preventing an acute disease or condition in a subject in need thereof, the method comprising administering to the subject a mesenchymal stem cell (MSC) contacted with a chemotherapy or conditioned media derived from the MSC contacted with the chemotherapy, thereby treating, ameliorating or preventing an acute disease or condition in a subject.

By another aspect, there is provided a method of treating, ameliorating or preventing an acute disease or condition in a subject in need thereof, the method comprising administering to the subject a mesenchymal stem cell (MSC) contacted with paclitaxel or conditioned media derived from the MSC contacted with paclitaxel, thereby treating, ameliorating or preventing an acute disease or condition in a subject.

By another aspect, there is provided a method of treating, ameliorating or preventing a systemic inflammatory response syndrome (SIRS) in a subject in need thereof, the method comprising administering to the subject a mesenchymal stem cell (MSC) contacted with a chemotherapy or conditioned media derived from the MSC contacted with the chemotherapy, thereby treating, ameliorating or preventing a SIRS in a subject.

By another aspect, there is provided a method of treating, ameliorating or preventing a systemic inflammatory response syndrome (SIRS) in a subject in need thereof, the method comprising administering to the subject a mesenchymal stem cell (MSC) contacted with paclitaxel or conditioned media derived from the MSC contacted with paclitaxel, thereby treating, ameliorating or preventing a SIRS in a subject.

By another aspect, there is provided a method of treating, ameliorating or preventing a systemic inflammatory response syndrome (SIRS) in a subject in need thereof, the method comprising administering to the subject a mesenchymal stem cell (MSC) contacted with cisplatin or conditioned media derived from the MSC contacted with cisplatin, thereby treating, ameliorating or preventing a SIRS in a subject.

In some embodiments of the invention, there is provided a method of treating, ameliorating or preventing an acute condition or disease in a subject in need comprising the step of administering to the subject in need a pharmaceutical composition comprising an effective amount (which may be therapeutically and/or prophylactic effective amount) of conditioned medium (CM) derived from a cell culture of repairing cells that were treated by an anticancer medicament.

In some embodiments of the invention, there is provided a method of treating, ameliorating or preventing an acute condition or disease in a subject in need comprising the step of administering to the subject in need a pharmaceutical composition comprising an effective amount (which may be therapeutically and/or prophylactic effective amount) of repairing cells that were treated by an anticancer medicament.

In some embodiments of the invention, there is provided a method of treating, ameliorating or preventing acute respiratory disease or an acute respiratory condition in a subject in need thereof comprising the step of administering to the subject in need thereof a pharmaceutical composition comprising an effective amount (therapeutically and/or prophylactic effective amount) of conditioned medium (CM) derived from a cell culture of repairing cells that were treated by an anticancer medicament. In some embodiments, the anticancer medicament is paclitaxel. In some embodiments, the anticancer medicament is cisplatin.

In some embodiments of the invention, there is provided a method of treating, ameliorating or preventing an acute respiratory disease or an acute respiratory condition in a subject in need thereof comprising the step of administering to the subject in need thereof a pharmaceutical composition comprising an effective amount (therapeutically and/or prophylactic effective amount) of repairing cells that were treated by an anticancer medicament. In some embodiments, the anticancer medicament is paclitaxel. In some embodiments, the anticancer medicament is cisplatin.

In some embodiments, the method is a method of treating. In some embodiments, the method is a method of ameliorating. In some embodiments, the method is a method of preventing. In some embodiments, the method is a therapeutic method.

By another aspect, there is provided an MSC contacted with paclitaxel or CM from the MSC contacted with paclitaxel for use in treating, ameliorating or preventing an acute disease or condition.

By another aspect, there is provided an MSC contacted with paclitaxel or CM from the MSC contacted with paclitaxel for use in treating, ameliorating or preventing an acute respiratory disease or conditions.

By another aspect, there is provided an MSC contacted with paclitaxel or CM from the MSC contacted with paclitaxel for use in treating, ameliorating or preventing SIRS.

As used herein, the terms “chemotherapy”, “chemotherapy agent” and “chemotherapy drug” are synonymous and used interchangeably. These terms refer to anticancer drugs or compounds or combinations thereof that are toxic to actively dividing cells and in particular cancer cells. Chemotherapy may inhibit mitosis, induce DNA damage or otherwise kill actively dividing cells. Examples of chemotherapies are well known in the art and any such compounds or combinations of compounds maybe used to active the MSCs. Examples of chemotherapies include, but are not limited to paclitaxel, cisplatin, gemcitabine, 5-fluorouracil, dacarbazine, doxorubicin, cyclophosphamide, methotrexate, docetaxel, vinorelbine, epirubicin, and many others. Examples of chemotherapies for activating MSCs can also be found in International Patent Application WO2020049552 herein incorporated by reference in its entirety. In some embodiments, the chemotherapy is selected from paclitaxel, cisplatin and gemcitabine. In some embodiments, the chemotherapy is selected from paclitaxel and cisplatin. In some embodiments, the chemotherapy is paclitaxel. In some embodiments, the chemotherapy is cisplatin. In some embodiments, the chemotherapy is gemcitabine.

In some embodiments, a MSC is administered. In some embodiments, a composition is administered. In some embodiments, the composition is a pharmaceutical composition. In some embodiments, the pharmaceutical composition comprises the MSC. In some embodiments, the pharmaceutical composition comprises the CM. In some embodiments, the pharmaceutical composition comprises the MSC and the CM. In some embodiment, the pharmaceutical composition is provided for the hereinabove recited uses. In some embodiments, a pharmaceutical composition a pharmaceutically acceptable carrier, excipient or adjuvant.

As used herein, the term “carrier,” “excipient,” or “adjuvant” refers to any component of a pharmaceutical composition that is not the active agent. As used herein, the term “pharmaceutically acceptable carrier” refers to non-toxic, inert solid, semi-solid liquid filler, diluent, encapsulating material, formulation auxiliary of any type, or simply a sterile aqueous medium, such as saline. Some examples of the materials that can serve as pharmaceutically acceptable carriers are sugars, such as lactose, glucose and sucrose, starches such as corn starch and potato starch, cellulose and its derivatives such as sodium carboxymethyl cellulose, ethyl cellulose and cellulose acetate; powdered tragacanth; malt, gelatin, talc; excipients such as cocoa butter and suppository waxes; oils such as peanut oil, cottonseed oil, safflower oil, sesame oil, olive oil, corn oil and soybean oil; glycols, such as propylene glycol, polyols such as glycerin, sorbitol, mannitol and polyethylene glycol; esters such as ethyl oleate and ethyl laurate, agar; buffering agents such as magnesium hydroxide and aluminum hydroxide; alginic acid; pyrogen-free water; isotonic saline, Ringer's solution; ethyl alcohol and phosphate buffer solutions, as well as other non-toxic compatible substances used in pharmaceutical formulations. Some non-limiting examples of substances which can serve as a carrier herein include sugar, starch, cellulose and its derivatives, powered tragacanth, malt, gelatin, talc, stearic acid, magnesium stearate, calcium sulfate, vegetable oils, polyols, alginic acid, pyrogen-free water, isotonic saline, phosphate buffer solutions, cocoa butter (suppository base), emulsifier as well as other non-toxic pharmaceutically compatible substances used in other pharmaceutical formulations. Wetting agents and lubricants such as sodium lauryl sulfate, as well as coloring agents, flavoring agents, excipients, stabilizers, antioxidants, and preservatives may also be present. Any non-toxic, inert, and effective carrier may be used to formulate the compositions contemplated herein. Suitable pharmaceutically acceptable carriers, excipients, and diluents in this regard are well known to those of skill in the art, such as those described in The Merck Index, Thirteenth Edition, Budavari et al., Eds., Merck & Co., Inc., Rahway, N.J. (2001); the CTFA (Cosmetic, Toiletry, and Fragrance Association) International Cosmetic Ingredient Dictionary and Handbook, Tenth Edition (2004); and the “Inactive Ingredient Guide,” U.S. Food and Drug Administration (FDA) Center for Drug Evaluation and Research (CDER) Office of Management, the contents of all of which are hereby incorporated by reference in their entirety. Examples of pharmaceutically acceptable excipients, carriers and diluents useful in the present compositions include distilled water, physiological saline, Ringer's solution, dextrose solution, Hank's solution, and DMSO. These additional inactive components, as well as effective formulations and administration procedures, are well known in the art and are described in standard textbooks, such as Goodman and Gillman's: The Pharmacological Bases of Therapeutics, 8th Ed., Gilman et al. Eds. Pergamon Press (1990); Remington's Pharmaceutical Sciences, 18th Ed., Mack Publishing Co., Easton, Pa. (1990); and Remington: The Science and Practice of Pharmacy, 21st Ed., Lippincott Williams & Wilkins, Philadelphia, Pa., (2005), each of which is incorporated by reference herein in its entirety. The presently described composition may also be contained in artificially created structures such as liposomes, ISCOMS, slow-releasing particles, and other vehicles which increase the half-life of the peptides or polypeptides in serum. Liposomes include emulsions, foams, micelles, insoluble monolayers, liquid crystals, phospholipid dispersions, lamellar layers and the like. Liposomes for use with the presently described peptides are formed from standard vesicle-forming lipids which generally include neutral and negatively charged phospholipids and a sterol, such as cholesterol. The selection of lipids is generally determined by considerations such as liposome size and stability in the blood. A variety of methods are available for preparing liposomes as reviewed, for example, by Coligan, J. E. et al, Current Protocols in Protein Science, 1999, John Wiley & Sons, Inc., New York, and see also U.S. Pat. Nos. 4,235,871, 4,501,728, 4,837,028, and 5,019,369.

The carrier may comprise, in total, from about 0.1% to about 99.99999% by weight of the pharmaceutical compositions presented herein.

In some embodiments, the pharmaceutical composition comprises an effective amount or number of MSCs. In some embodiments, the pharmaceutical composition comprises an effective amount of CM. In some embodiments, the pharmaceutical composition comprises an effective amount of MSCs and CM. In some embodiments, an effective amount is a therapeutically effective amount. In some embodiments, an effective amount is an amount sufficient to treat, prevent or ameliorate at least one symptom of the acute disease or condition or the cytokine storm.

The term “therapeutically effective amount” refers to an amount of an active compound effective to treat a disease or disorder in a mammal. The term “a therapeutically effective amount” refers to an amount effective, at dosages and for periods of time necessary, to achieve the desired therapeutic or prophylactic result. The exact dosage form and regimen would be determined by the physician according to the patient's condition.

Unless otherwise indicated, the term “effective amount” as referred to herein designate the quantity of a compound or a composition, which is sufficient to yield a desired response. For therapeutic purposes, the effective amount is also one in which any toxic or detrimental effects of the compound or the composition are outweighed by the therapeutically beneficial effects. The specific effective or sufficient amount will vary with such factors as the particular condition being treated, the physical condition of the patient (e.g., the patient's body mass, age, or gender), the type of mammal or animal being treated, the duration of the treatment, the nature of concurrent therapy (if any), and the specific formulations employed. Effective amount may be expressed, for example, in grams, milligrams or micrograms or as milligrams per kilogram of body weight (mg/Kg) in concentration of the active ingredients and the like. Alternatively, the effective amount can be expressed in the concentration of the active component, such as, molar concentration, mass concentration, volume concentration, molality, mole fraction, mass fraction and mixing ratio. Further, persons having ordinary skills in the related art could calculate the human equivalent dose (HED) for the medicament (such as the compound of the present disclosure) based on the doses determined from animal models. For example, one may follow the guidance for industry published by US Food and Drug Administration (FDA) entitled “Estimating the Maximum Safe Starting Dose in Initial Clinical Trials for Therapeutics in Adult Healthy Volunteers” in estimating a maximum safe dosage for use in human subjects.

Unless otherwise indicated, a “therapeutically effective amount” of a compound or a composition is an amount sufficient to provide a therapeutic benefit in the treatment or management of a disease or condition, or to delay or minimize one or more symptoms associated with the disease or condition. A therapeutically effective amount of a compound or a composition is an amount of one or more therapeutic agent, alone or in combination with other therapies, which provides a therapeutic benefit in the treatment or management of the disease or condition. The term “therapeutically effective amount” can encompass an amount that improves overall therapy, reduces or avoids symptoms or causes of a disease or condition, or enhances the therapeutic efficacy of another therapeutic agent.

Unless otherwise indicated, a “prophylactically effective amount” of a compound or a composition is an amount sufficient to prevent a disease or condition, or one or more symptoms associated with the disease or condition, or prevent its recurrence. A prophylactically effective amount of a compound or a composition means an amount of therapeutic agent, alone or in combination with other agents, which provides a prophylactic benefit in the prevention of the disease. The term “prophylactically effective amount” can encompass an amount that improves overall prophylaxis or enhances the prophylactic efficacy of another prophylactic agent.

In some embodiments, the acute respiratory disease or condition is a disease. In some embodiments, the acute respiratory disease or condition is a condition. In some embodiments, the acute respiratory disease or condition is selected from the group consisting of acute respiratory distress syndrome (ARDS) and acute lung injury (ALI). In some embodiments, the acute respiratory disease or condition is ARDS. In some embodiments, the acute respiratory disease or condition is not lung injury. In some embodiments, the acute respiratory disease or condition is acute lung injury. In some embodiments, acute lung injury is not chronic lung injury. In some embodiments, the acute lung injury does not comprise fibrosis. In some embodiments, the acute respiratory disease or condition does not comprise fibrosis. In some embodiments, the fibrosis is lung fibrosis.

In some embodiments, an acute disease is not a chronic disease. In some embodiments, an acute disease is of recent onset. In some embodiments, the acute respiratory disease or condition is of short duration. In some embodiments, short is less than 12 months, 11 months, 10 months, 9 months, 8 months, 7 months, 6 months, 5 months, 4 months, 3 months, 2 months, 1 month, 4 weeks, 3 weeks, 2 weeks or 1 week. Each possibility represents a separate embodiment of the invention. In some embodiments, recent is within less than 12 months, 11 months, 10 months, 9 months, 8 months, 7 months, 6 months, 5 months, 4 months, 3 months, 2 months, 1 month, 4 weeks, 3 weeks, 2 weeks or 1 week. Each possibility represents a separate embodiment of the invention. In some embodiments, an acute disease does not comprise fibrosis.

In some embodiments, the ARDS is developed from one or more conditions associated with severe lung trauma, viral or bacterial pneumonia, sepsis, aspiration of gastrointestinal contents with consequent pulmonary infection, corona virus disease or acute pancreatitis. In some embodiments, the acute respiratory disease or condition is selected from severe lung trauma, viral or bacterial pneumonia, sepsis, aspiration of gastrointestinal contents with consequent pulmonary infection, corona virus disease or acute pancreatitis. In some embodiments, the acute respiratory disease or condition is selected from severe lung trauma, sepsis, pulmonary infection, and acute pancreatitis. In some embodiments, the acute respiratory disease or condition is severe lung trauma. In some embodiments, the acute respiratory disease or condition is sepsis. In some embodiments, the acute respiratory disease or condition is pulmonary infection. In some embodiments, the acute respiratory disease or condition is acute pancreatitis. In some embodiments, the subject suffers from a pulmonary infection. In some embodiments, the acute respiratory disease or condition is a systemic inflammatory response syndrome (SIRS).

In some embodiments, the pulmonary infection is selected from a bacterial infection, a viral infection and an infection consequent to aspiration of gastrointestinal contents. In some embodiments, the pulmonary infection is a bacterial infection. In some embodiments, the pulmonary infection is a viral infection. In some embodiments, the pulmonary infection is an infection consequent to aspiration of gastrointestinal contents. In some embodiments, the infection is pneumonia. In some embodiments, the pulmonary infection causes SIRS. In some embodiments, the pulmonary infection causes cytokine storm. In some embodiments, the SIRS is cytokine storm. In some embodiments, the SIRS is cytokine release syndrome.

In some embodiments, the ARDS is associated with one or more of noncardiogenic pulmonary oedema, acute hypoxaemia or the need for mechanical ventilation. In some embodiments, the ARDS is associated with noncardiogenic pulmonary oedema. In some embodiments, the ARDS is associated with acute hypoxaemia. In some embodiments, the ARDS is associated with need for mechanical ventilation. In some embodiments, the ARDS is associated with SIRS. In some embodiments, the ARDS is associated with cytokine storm.

In some embodiments, the viral infection is a coronavirus infection. In some embodiments, the disease is corona virus infection. In some embodiments, the Corona virus disease is COVID 19 disease, HCoV-NL63, HCoV-OC43, HCoV-229E, HCoV-HKUI, SARS-CoV (severe acute respiratory syndrome coronavirus), CoV MERS (middle east respiratory syndrome coronavirus) or SARS-CoV-2. In some embodiments, the coronavirus is selected from human coronavirus (HcoV)-NL63, HCoV-OC43, HCoV-229E, HCoV-HKUI, severe acute respiratory syndrome coronavirus (SARS-CoV-1), middle east respiratory syndrome coronavirus (MERS-CoV) and SARS-CoV-2. In some embodiments, the coronavirus is SARS-CoV-1. In some embodiments, the coronavirus is SARS-CoV-2. In some embodiments, the coronavirus is MERS-CoV.

In some embodiments, the subject is a mammal. In some embodiments, the subject is a human. In some embodiments, the subject is a subject susceptible to coronavirus infection. In some embodiments, the subject is avian. In some embodiments, the subject is feline. In some embodiments, the subject is a primate.

In some embodiments, the subject is infected by a coronavirus. In some embodiments, the subject has a confirmed coronavirus infection. Coronavirus infection can be confirmed by any method known in the art. Commonly diagnosis is performed by a PCR test. Methods for performing PCR testing are known in the art and can be found for example in WHO interim guidance 19 Mar. 2020: Laboratory testing for coronavirus disease (COVID-19) in suspected human cases (apps.who.int/iris/rest/bitstreams/1271387/retrieve), herein incorporated by reference in its entirety.

In some embodiments, the subject is inflicted with a severe COVID 19 disease. A severe Corona Virus Disease/infection may include a subject that has one or more of the following conditions: a cytokine storm (aka cytokine release syndrome, or CRS), secondary hemophagocytic lymphohistiocytosis (sHLH), an acute respiratory distress syndrome (ARDS), unremitting fever, cytopenia, and hyperferritinaemia. In some embodiments, the subject is at risk of developing severe Corona Virus Disease, i.e., is at risk of developing one or more of the following conditions: a cytokine storm (aka cytokine release syndrome, or CRS), secondary hemophagocytic lymphohistiocytosis (sHLH), an acute respiratory distress syndrome (ARDS), unremitting fever, cytopenia, and hyperferritinaemia. In some embodiments, the treatment ameliorates, cures or reduces at least one of the following symptoms: a cytokine storm (CCS), secondary hemophagocytic lymphohistiocytosis (sHLH), an acute respiratory distress syndrome (ARDS), unremitting fever, cytopenias, and hyperferritinaemia.

In some embodiments, the infection is a severe infection. In some embodiments, the infection is pneumonia. In some embodiments, the subject is in the first phase of coronavirus infection. In some embodiments, the first phase is the first stage. In some embodiments, the first phase is early infection. In some embodiments, the first phase is pre-symptomatic. In some embodiments, the first phase comprises upper respiratory tract infection. In some embodiments, the first phase comprises upper respiratory tract symptoms. In some embodiments, the subject is in the second phase of coronavirus infection. In some embodiments, the second phase is the second stage. In some embodiments, the second phase is the pulmonary phase. In some embodiments, the second stage comprises two parts IIA and IIB. In some embodiments, stage IIA comprises pneumonia without hypoxia. In some embodiments, stage IIB comprises pneumonia with hypoxia. In some embodiments, the second phase comprises lower respiratory tract infection. In some embodiments, the second stage comprises pneumonia. In some embodiments, the subject is in either phase 1 or phase 2.

In some embodiments, the subject is in phase 3. In some embodiments, phase 3 is stage 3. In some embodiments, stage 3 is the third stage. In some embodiments, phase 3 is the hyperinflammation phase. In some embodiments, phase 3 comprises cytokine storm. In some embodiments, phase 3 comprises ARDS. In some embodiments, phase 3 comprises ICU entrance. In some embodiments, phase 3 comprises mechanical ventilation. In some embodiments, phase 3 is the acute phase of infection. In some embodiments, the subject has entered an acute phase of infection. In some embodiments, stage 3 is a severe stage of infection.

In some embodiments, the subject is at risk of a coronavirus infection. In some embodiments, a subject at risk is a non-vaccinated subject. In some embodiments, a subject at risk is a subject with an immunodeficiency. In some embodiments, a subject at risk is a subject with at least one comorbidity. In some embodiments, a subject at risk is a subject in a region/location with a high infection rate. In some embodiments, a high infection rate is a rate of infection above 1, 2, 3, 4, 5, 6, 7, 8, 9, 10, 11, 12, 13, 14, 15, 16, 17, 18, 19 or 20% infection. Each possibility represents a separate embodiment of the invention. In some embodiments, a high infection rate is a rate of infection above 10% infection. In some embodiments, a subject at risk is a front-line worker. In some embodiments, a subject at risk is a medical worker. In some embodiments, a subject at risk is a nursing home worker. In some embodiments, a subject at risk is a subject who cannot be vaccinated. In some embodiments, a subject at risk is anyone during a pandemic. In some embodiments, the subject does not suffer from a comorbidity.

In some embodiments, the subject suffers from at least one comorbidity. In some embodiments, comorbidity is comorbidity with the coronavirus. In some embodiments, the comorbidity is selected from hypertension, diabetes mellitus, coronary heart disease, cerebrovascular diseases, obesity, dyslipidemia, asthma, chronic obstructive pulmonary disease (COPD), chromic liver disease, and chronic kidney diseases. In some embodiments, the comorbidity is hypertension. In some embodiments, the comorbidity is diabetes mellitus. In some embodiments, the comorbidity is coronary heart disease. In some embodiments, the comorbidity is cerebrovascular disease. In some embodiments, the comorbidity is obesity. In some embodiments, the comorbidity is dyslipidemia. In some embodiments, the comorbidity is asthma. In some embodiments, the comorbidity is COPD. In some embodiments, the comorbidity is chromic liver disease. In some embodiments, the comorbidity is chronic kidney disease.

In some embodiments, the subject has one or more of the following conditions: a cytokine storm (aka cytokine release syndrome, or CRS), secondary hemophagocytic lymphohistiocytosis (sHLH), an acute respiratory distress syndrome (ARDS), unremitting fever, cytopenia, and hyperferritinaemia. In some embodiments, the subject suffers from a cytokine storm. In some embodiments, the subject suffers from cytokine release syndrome. In some embodiments, the subject suffers from sHLH. In some embodiments, the subject suffers from ARDS. In some embodiments, the subject suffers from unremitting fever. In some embodiments, the subject suffers from cytopenia. In some embodiments, the subject suffers from hyperferritinaemia.

As used herein, the term “cytokine release syndrome” (CRS) refers to systemic inflammatory response that is triggered in response to a stimulus or stimuli. Cytokine release syndrome can have cytokine storm characteristics, but a cytokine storm is the immediate onset of CRS in response to a stimulus. It should be noted that severe CRS can be called a cytokine storm and so for the most part the terms can be used interchangeably. These conditions occurs when a large number of lymphocytes are activated and release proinflammatory cytokines systemically. This leads to a cascade of lymphocyte activation and cytokine production. Cytokine storm often leads to organ failure and is frequently deadly. Methods of identifying cytokine storm are well known in the art and any such method can be used. Commonly a blood draw and identification of the presence of high levels of circulating proinflammatory cytokines is carrier out. In some embodiments, the subject suffers from CRS. In some embodiments, the subject suffers from cytokine storm.

As used herein, the terms “treatment” or “treating” of a disease, disorder, or condition encompasses alleviation of at least one symptom thereof, a reduction in the severity thereof, or inhibition of the progression thereof. Treatment need not mean that the disease, disorder, or condition is totally cured. To be an effective treatment, a useful composition or method herein needs only to reduce the severity of a disease, disorder, or condition, reduce the severity of symptoms associated therewith, or provide improvement to a patient or subject's quality of life.

In some embodiments, the treatment ameliorates, cures, reduces or prevents at least one of the following conditions: a cytokine storm (CCS), secondary hemophagocytic lymphohistiocytosis (sHLH), an acute respiratory distress syndrome (ARDS), unremitting fever, cytopenias, and hyperferritinaemia. In some embodiments, treatment comprises treating at least one symptom. In some embodiments, treatment comprises treating cytokine storm. In some embodiments, treatment comprises reducing inflammation. In some embodiments, treatment comprises treating the infection. In some embodiments, treatment comprises treating sHLH. In some embodiments, treatment comprises treating ARDS. In some embodiments, treatment comprises treating unremitting fever. In some embodiments, treatment comprises treating cytopenia. In some embodiments, treatment comprises treating hyperferritanaemia.

In some embodiments, the subject is at risk of developing a severe COVID 19 disease and has the risk of developing one or more of the following conditions: a cytokine storm (aka cytokine release syndrome, or CRS), secondary hemophagocytic lymphohistiocytosis (sHLH), an acute respiratory distress syndrome (ARDS), unremitting fever, cytopenia, and hyperferritinaemia.

In some embodiments, the treatment prevents at least one of the following symptoms: a cytokine storm (aka cytokine release syndrome, or CRS), secondary hemophagocytic lymphohistiocytosis (sHLH), an acute respiratory distress syndrome (ARDS), unremitting fever, cytopenias, and hyperferritinaemia. In some embodiments, preventing comprises preventing at least one symptom. In some embodiments, preventing comprises preventing cytokine storm. In some embodiments, preventing comprises preventing an increase in inflammation. In some embodiments, preventing comprises preventing the infection. In some embodiments, preventing comprises preventing symptomatic disease. In some embodiments, preventing comprises preventing sHLH. In some embodiments, preventing comprises preventing ARDS. In some embodiments, preventing comprises preventing unremitting fever. In some embodiments, preventing comprises preventing cytopenia. In some embodiments, preventing comprises preventing hyperferritanaemia.

In some embodiments, the method comprises administering the MSCs treated with paclitaxel. In some embodiments, the method comprises administering the CM from MSCs treated with paclitaxel. In some embodiments, the method comprises administering the MSC and their CM. In some embodiments, the administering is administering a composition. In some embodiments, the composition is devoid of the anticancer medicament. In some embodiments, the composition is devoid of paclitaxel. In some embodiments, the composition is configured for systemic administration. In some embodiments, the composition is configured for local administration. In some embodiments, local is locally to the lungs. In some embodiments, local is intratracheal. In some embodiments, local is directly to the lungs. In some embodiments, administration to the lungs comprise inhalation. In some embodiments, the composition is configured for administration to the lungs. In some embodiments, the composition is configured as an aerosol.

As used herein, the terms “administering,” “administration,” and like terms refer to any method which, in sound medical practice, delivers a composition containing an active agent to a subject in such a manner as to provide a therapeutic effect. One aspect of the present subject matter provides for intratracheal administration of a therapeutically effective amount of a composition of the present subject matter to a patient in need thereof. Other suitable routes of administration can include parenteral, oral, subcutaneous, intravenous, intramuscular, inhalation or intraperitoneal. In some embodiments, the administration is systemic. In some embodiments, the administration is local. In some embodiments, the administration is inhalation.

The dosage administered will be dependent upon the age, health, and weight of the recipient, kind of concurrent treatment, if any, frequency of treatment, and the nature of the effect desired.

Methods of producing the MSCs and CM of the invention are provided herein below. In some embodiments, the contacting with the paclitaxel is for at least 10 minutes, 15 minutes, 20 minutes, 25 minutes, 30 minutes, 35 minutes, 40 minutes, 45 minutes, 50 minutes, 55 minutes, 1 hour, 2 hours, 3 hours, 4 hours, 5 hours, 6 hours, 8 hours, 12 hours, 16 hours, 18 hours, 24 hours, 36 hours, 48 hours or 72 hours. Each possibility represents a separate embodiment of the invention. In some embodiments, the contacting with the paclitaxel is for at least 24 hours. In some embodiments, the contacting with the paclitaxel is for at most 24, 36, 48, 60, 72, 84, 96, 120, 144 or 168 hours. Each possibility represents a separate embodiment of the invention. In some embodiments, the contacting was for about 24 hours.

In some embodiments, the contacting is in culture. In some embodiments, the contacting is ex vivo. In some embodiments, the contacting is in vitro. In some embodiments, the contacting is transient. In some embodiments, the contacting is followed by removal of the media comprising the anticancer medicament (i.e., paclitaxel). In some embodiments, new media is added, and the new media is after used as the conditioned media. In some embodiments, conditioned media is from a culture of the MSC for at least 12, 16, 18, 24, 48, 60, 72, 84, or 96 hours. Each possibility represents a separate embodiment of the invention. In some embodiments, conditioned media is from a culture of the MSC for at least 24 hours. In some embodiments, conditioned media is from a culture of the MSC for at most 72, 84, 96, 108, 120, 132, 144, 156, or 168 hours. Each possibility represents a separate embodiment of the invention. In some embodiments, conditioned media is from a culture of the MSC for about 24 hours.

In some embodiments, the MSCs are human MSCs. In some embodiments, the MSCs are mammalian MSCs. In some embodiments, the MSCs are derived from bone marrow, adipose, dental pulp, umbilical cord, or placenta. In some embodiments, the MSCs are derived from bone marrow. Methods of identifying and isolating MSCs are well known in the art and provided hereinbelow. Any such method may be employed for isolated the MSCs to be contacted with the anticancer medicament (i.e., paclitaxel).

In some embodiments, the method further comprising administrating an additional drug or therapy.

In some embodiments, the additional drug or therapy is an anti-cytokine therapy, anti-IL-6, anti-IL-1, an anti-viral drug, interferon-alpha2b, analgesic drug, corticosteroid or mechanical ventilation. The anti-viral drug may be in some embodiments, remdesivir, ribavirin, oseltamivir or zanamivir. In some embodiments, the additional drug or therapy is selected from: an anti-cytokine therapy, an anti-viral drug, an analgesic drug, a corticosteroid and mechanical ventilation. In some embodiments, the anti-viral drug is selected from remdesivir, ribavirin, oseltamivir, and zanamivir. In some embodiments, the anti-viral drug is selected from remdesivir, ribavirin, oseltamivir, zanamivir and interferon-alpha 2b. In some embodiments, the additional drug or therapy is interferon-alpha 2b. In some embodiments, the anti-cytokine therapy is selected from anti-IL6, anti-IL6R and anti-IL1. In some embodiments, the anti-cytokine therapy is anti-IL6. In some embodiments, the anti-cytokine therapy is anti-IL6R. In some embodiments, the anti-cytokine therapy is anti-IL2.

In some embodiments, the treatment is associated with reduction of one or more pro-inflammatory cytokines, such as, IL-6 (normal: <7 pg/ml; ARDS: >400 pg/ml), TNFα (normal: <5 pg/ml; ARDS: 200-400 pg/ml), IL-2 (normal: <5 pg/ml; ARDS: 200-400 pg/ml), G-CSF (normal <20 pg/ml; ARDS: 200-2000 pg/ml), MIP-1α (normal <100 pg/ml; ARDS: 200-2000 pg/ml) or INFγ (normal: <15 pg/ml; ARDS: 50-10000 pg/ml) and/or improvement in respiratory parameters, such as O2 saturation (SpO2) and respiratory rate.

In some embodiments, the treatment comprises reduction of a proinflammatory cytokine. In some embodiments, the treatment comprises reduction of at least one proinflammatory cytokine. In some embodiments, the treatment comprises reduction of at least two proinflammatory cytokines. In some embodiments, the proinflammatory cytokine is selected from IL-6, TNFα, IL-2, IL-3, G-CSF, MIP-1α, and INFγ. In some embodiments, the proinflammatory cytokine is IL-6. In some embodiments, the proinflammatory cytokine is TNFα. In some embodiments, the proinflammatory cytokine is IL-2. In some embodiments, the proinflammatory cytokine is IL-3. In some embodiments, the proinflammatory cytokine is G-CSF. In some embodiments, the proinflammatory cytokine is MIP-1α. In some embodiments, the proinflammatory cytokine is INFγ.

In some embodiments, treatment comprises improvement in a respiratory parameter. In some embodiments, the parameter is respiratory rate. In some embodiments, the parameter is inhalation volume.

In some embodiments, treatment comprises an increase in at least one anti-inflammatory cytokine. In some embodiments, treatment comprises an increase in at least two anti-inflammatory cytokines. In some embodiments, the anti-inflammatory cytokine is selected from IL-10 and FGF acidic. In some embodiments, the anti-inflammatory cytokine is IL-10. In some embodiments, the anti-inflammatory cytokine is FGF acidic.

In some embodiments, treatment comprises an alteration in immune cell infiltration. In some embodiments, the infiltration is to a site of acute inflammation. In some embodiments, the infiltration is to a site of infection. In some embodiments, the infiltration is to the lungs. In some embodiments, the alteration comprises at least one of decrease of CD4 helper cells, increase of B cells, increase of cytotoxic T cells, increase in myeloid derived suppressor cells (MDSCs) and increase in M2 macrophages. In some embodiments, the alteration comprises at least one of decrease of CD4 helper cells, increase of B cells, increase of cytotoxic T cells, and increase in myeloid derived suppressor cells (MDSCs). In some embodiments, the alteration is a decrease in CD4 helper cells. In some embodiments, the alteration is an increase in B cells. In some embodiments, the alteration is an increase in cytotoxic T cells. In some embodiments, the alteration is an increase in MDSCs. In some embodiments, the alteration is an increase in M2 macrophages. In some embodiments, M2 are anti-inflammatory macrophages. In some embodiments, CD4 T helper cells are effector cells. In some embodiments, effector cells are activated effector cells. In some embodiments, cytotoxic T cells are CD8 T cells. In some embodiments, cytotoxic T cells are activated T cells.

In some embodiments, the increase in in the subject. In some embodiments, the decrease is in the subject. In some embodiments, the alteration is in the subject. In some embodiments, the improvement is in the subject. In some embodiments, in the subject is in the blood of the subject. In some embodiments, the increase, decrease, alteration and/or improvement are as compared to a subject not treated. In some embodiments, the increase, decrease, alteration and/or improvement are as compared to a subject treated with an MSC not contacted or its CM. In some embodiments, the increase, decrease, alteration and/or improvement are as compared to a subject treated with an MSC contacted with an anticancer medicament other than paclitaxel or its CM. In some embodiments, an anticancer medicament other than paclitaxel is cisplatin. In some embodiments, an anticancer medicament other than paclitaxel is gemcitabine. In some embodiments the increase, decrease, alteration and/or improvement are significant. In some embodiments, significant is statistically significant.

In some embodiments, prior to the treatment the subject has elevated ferritin (normal: male—12-300 ng/ml, female—12-150 ng/ml; ARDS: males—270 ng/ml, female—680 ng/ml) and/or IL-6 (normal: <7 pg/ml; ARDS: >400 pg/ml) in the blood. In some embodiments, the treatment decreases IL-6 level to normal levels, i.e. <7 pg/ml. In some embodiments, the treatment substantially decreases the level of IL-6 in a patient by up to four-fold. In some embodiments, the treatment substantially decreases the level of IL-6 in a patient by up to two-fold. In some embodiments, the treatment substantially decreases the level of IL-6 in a patient by to two-fold or more.

In some embodiments, the subject has elevated increased the level of one or more of the following: interleukin (IL)-2, IL-3, granulocyte-colony stimulating factor (GCSF), and other pro-inflammatory cytokines. In some embodiments, the treatment decreases the level of at least one pro-inflammatory cytokine to normal levels. In some embodiments, the treatment decreases the level of at least one pro-inflammatory cytokine in a patient by more than two-fold. In some embodiments, the treatment decreases the level of at least one pro-inflammatory cytokine in a patient by more than four-fold. In some embodiments, the subject has elevated increased interleukin (IL)-2, IL-3, granulocyte-colony stimulating factor (GCSF), and other pro-inflammatory cytokines.

In some embodiments, the anticancer drug is paclitaxel or cisplatin. In some embodiments, the anticancer drug is paclitaxel. In some embodiments, the anticancer drug is cisplatin. In some embodiments, the anticancer drug is not cisplatin. In some embodiments, the anticancer drug is not gemcitabine.

In some embodiments, the composition is administered by oral, sublingual, subcutaneous, intramuscular, intravenous, topical, local, intratracheal, intranasal, transdermal and rectal administration. In some embodiments, the composition is administered by systemic administration. In some embodiments, systemic administered is intravenous (iv) administration. In some embodiments, the composition is administered by local administration. In some embodiments, local administration is intratracheal administration.

In some embodiments, the composition is administered by intratracheal or intravenous (iv) administration. In some embodiments the composition may be administered by continuous infusion. In some embodiments, mini-pumps may be used.

As used herein, “preventing” or “prevention” or “inhibit” or “inhibition” “prophylactic” interchangeably refers to the reduction of likelihood of the risk of acquiring a condition that may be fatal to the subject (for example, when the subject does not yet experience or display all symptoms of the disease, or the symptoms are minor, the outburst of the disease will be reduced or inhibited). Biological and physiological parameters for identifying such patients are well known to physicians and depend on the disease or disorder. In the case of severe Covid 19 or ARDS the preventive treatment will be given once the patient shows a cytokine storm (aka cytokine release syndrome, or CRS), secondary hemophagocytic lymphohistiocytosis (sHLH), unremitting fever, cytopenias, hyperferritinaemia or elevated levels of pro-inflammatory cytokines in the plasma such as IL-6, TNFα, IL-2, IL-3, G-CSF, MIP-1α or INFγ, reduction in respiratory parameters any combination thereof. In some embodiments, the treatment for the prevention of severe ARDS may start when the patient has one or more of more than 30 respirations per minutes, less than 93% blood oxygen saturation, and more than 120 heart beat rate per minute.

The terms “treatment” or “treating” of a subject includes the application or administration of the secreted cell products of the repairing cells or the repairing cells themselves to a subject with the purpose of stabilizing, curing, healing, alleviating, relieving, altering, remedying, less worsening, ameliorating, improving, or affecting the disease or condition, the symptom of the disease or condition, or the risk of (or susceptibility to) the disease or condition. The term refers to any indicia of success in the treatment or amelioration of acute condition, pathology including any objective or subjective parameter such as abatement; remission; lessening of the rate of worsening; lessening severity of the disease; stabilization, diminishing of symptoms or making the injury, pathology or condition more tolerable to the subject; slowing in the rate of deteriorating or decline. In some embodiments, the term “treating” can include increasing a subject's life expectancy and/or delay before additional treatments are required and or prevent death.

“Repairing cells” or “repairing mesenchymal stem cells” or “repairing macrophage cells” or “repairing fibroblast cells” and the like, refer herein to cells, mesenchymal stem cells or macrophages or other cells that were previously educated with an anticancer agent for a period of at least 10, 20, 30, 40, 50 minutes or an hour, 2, 3, 4, 5, 6, 7, 8, 9, 10, 12, 14, 16, 18, 20, 22 24, 48 or 72 hours or more using standard medium. These repairing cells or the conditioned media therefrom may be used in the methods of treating described herein. Examples for such an anticancer agent is one or more of Gemcitabine, Paclitaxel, Paclitaxel and Anakinra, Cisplatin, 5-FU, Dacarbazine, Temozoloamine targeted drugs (e.g. Bortezomib), radiation, antiangiogenic drugs and immune checkpoint inhibitors antibodies. The term “activating composition” or “repairing composition” includes either cells that are activated by an anticancer therapy or the conditioned media thereof, or both. The conditioned media may be diluted, concentrated, dried or lyophilized. The activated cells are any of the following: activated mesenchymal stem cells and/or activated macrophages and/or T cells and/or fibroblasts and/or neural tissue cells (such as astrocytes and glia cells) and/or adipose tissue-derived cells and/or other tissues resident cells or any other activated cell that can be used for treating acute conditions, diseases or disorders, such as, ARDS or severe Corona virus disease. The number of the activated MSC or the any other activated cell or repairing cells of the invention, or the conditioned medium produced therefrom, the administration dosage form and the dosing regimen as well as the location in case of a local administration depend on the type of the acute disease, condition, or disorder, the location of organ to be treated and the severity of the disease. In most cases, but not exclusively, the conditioned medium is produced from at least 10⁵-10⁶ cells per 1 ml. In some embodiments, the treatment is by using at least between 1-10×10⁶ cells, per treatment. In some embodiments, the repairing composition, which is the pharmaceutical composition of the invention includes repairing cells that were previously activated by an anticancer, or the CM thereof; and a pharmaceutically acceptable carrier, may be administered once a day, every other day, every three days or once a week until the patient is recovered. In some embodiments, the repairing cells or the CM thereof may be administered to a patient that recovered from ARDS, AlI, CRS or from Corona Virus Disease on a routine basis in order to prevent late damages to the lungs or to other body organs.

It should be noted that in some embodiments of the invention, the conditioned medium can be concentrated by centrifugation at, for example, 1100,000 g to obtain exosomes and other products. In some cases, the conditioned medium can be lyophilized in order to keep it as dry product for a later use in reconstitution, for example. It is noted that the in the method of treatment of the invention, the term “conditioned medium” or “conditioned media” or “CM” refers also to the secretome, the dry product, the supernatant, as well as to untouched or diluted conditioned media.

In an embodiment of the invention, there is provided an “activating composition” also interchangeably termed here “repairing composition”, as hereinafter defined. According to some embodiments of the invention the activating composition comprises mesenchymal stem cells and/or macrophages and/or T cells and/or fibroblasts that were activated by an anticancer agent, such as a chemotherapy agent. In other embodiments, the activating composition comprises the supernatant or the conditioned media of a preparation that includes a mesenchymal stem cell and/or macrophages and/or T cells and/or fibroblasts that were activated by a specific anticancer agent such as a chemotherapy agent and that was separated, for example, by centrifugation, using the secretome of such activated cells. The term “secretome” refers to proteins, metabolites, enzymes, lipids, sugar molecules, and extracellular vesicles, such as exosomes that are secreted by cells into the extracellular space. In some embodiments of the invention the cells may be collected from the patient, treated with an anticancer and returned to the patient. In some embodiments of the invention the cells may be collected from the patient, treated with an anticancer and the CM thereof may be administered to the patient.

“Extracellular vesicles” “exosomes” are blubbing or secretion of small vesicles from cells which contains proteins, miRNA, RNA, DNA and other biological materials which can be transferred between cells. These vesicles have been shown to act in physiological and pathological conditions.

In some embodiments of the invention, there is provided an activating composition comprising mesenchymal stem cells and/or macrophages that is activated by a chemotherapy agent or other anti-cancer agent, wherein the mesenchymal stem cells and/or macrophages and/or T cells and/or fibroblasts that are activated by a chemotherapy agent are separated from the conditioned medium and wherein the conditioned medium is used as a therapy. In some embodiments, the anti-cancer agent is paclitaxel. In some embodiments, the anticancer agent is cisplatin.

The routes for administration of the composition of the invention include oral (e.g., as a tablet, capsule, or as an ingestible solution), topical, mucosal (e.g., as a nasal spray or aerosol for inhalation), nasal, parenteral (e.g., by an injectable form), gastrointestinal, intraspinal, intraperitoneal, intramuscular, intravenous, intrauterine, intraocular, intradermal, intracranial, intratracheal, intravaginal, intracerebroventricular, intracerebral, subcutaneous, ophthalmic (including intravitreal or intracameral), transdermal, rectal, buccal, epidural and sublingual. The compositions of the invention may be especially formulated for any of those administration routes.

There may be different composition/formulation requirements depending on the different delivery systems. It is to be understood that not all of the compositions need to be administered by the same route. Likewise, if the composition comprises more than one active component, then those components may be administered by different routes. By way of example, the pharmaceutical composition of the invention may be formulated to be delivered using a mini-pump or by a mucosal route, for example, as a nasal spray or aerosol for inhalation or ingestible solution, or parenterally in which the composition is formulated by an injectable form, for delivery, by, for example, an intravenous, intramuscular or subcutaneous route. Alternatively, the formulation may be designed to be delivered by multiple routes.

In some embodiments, the formulation may be administered as enteric coated formulations. As enteric coating layer polymers, one or more, separately or in combination, of the following can be used; e.g., solutions or dispersions of methacrylic acid copolymers, cellulose acetate phthalate, cellulose acetate butyrate, hydroxypropyl methylcellulose phthalate, hydroxypropyl methylcellulose acetate succinate, polyvinyl acetate phthalate, cellulose acetate trimellitate, carboxymethylethylcellulose, shellac or other suitable enteric coating layer polymer(s).

When appropriate, the pharmaceutical compositions can be administered by inhalation, in the form of a suppository or pessary, topically in the form of a lotion, solution, cream, ointment or dusting powder, by use of a skin patch, orally in the form of tablets containing excipients such as starch or lactose, or in capsules or ovules either alone or in admixture with excipients, or in the form of elixirs, solutions or suspensions containing flavouring or colouring agents, or they can be injected parenterally, for example intravenously, intramuscularly or subcutaneously. For buccal or sublingual administration, the compositions may be administered in the form of tablets or lozenges, which can be formulated in a conventional manner.

When the composition of the invention is to be administered parenterally, such administration includes one or more of: intravenously, intraarterially, intraperitoneally, intrathecally, intraventricularly, intraurethrally, intrasternally, intracranially, intramuscularly or subcutaneously administering the agent; and/or by using infusion techniques.

Pharmaceutical compositions of the invention can be administered parenterally, e.g., by infusion or injection. Pharmaceutical compositions suitable for injection or infusion may be in the form of a sterile aqueous solution, a dispersion or a sterile powder that contains the active ingredient, adjusted, if necessary, for preparation of such a sterile solution or dispersion suitable for infusion or injection. This preparation may optionally be encapsulated into liposomes. In all cases, the final preparation must be sterile, liquid, and stable under production and storage conditions. To improve storage stability, such preparations may also contain a preservative to prevent the growth of microorganisms. Prevention of the action of micro-organisms can be achieved by the addition of various antibacterial and antifungal agents, e.g., paraben, chlorobutanol, or ascorbic acid. In many cases isotonic substances are recommended, e.g., sugars, buffers and sodium chloride to assure osmotic pressure similar to those of body fluids, particularly blood. Prolonged absorption of such injectable mixtures can be achieved by introduction of absorption-delaying agents, such as aluminium monostearate or gelatin.

Dispersions can be prepared in a liquid carrier or intermediate, such as glycerine, liquid polyethylene glycols, triacetin oils, and mixtures thereof. The liquid carrier or intermediate can be a solvent or liquid dispersive medium that contains, for example, water, ethanol, a polyol (e.g., glycerol, propylene glycol or the like), vegetable oils, non-toxic glycerine esters and suitable mixtures thereof. Suitable flowability may be maintained, by generation of liposomes, administration of a suitable particle size in the case of dispersions, or by the addition of surfactants.

For parenteral administration, the composition is best used in the form of a sterile aqueous solution, which may contain other substances, for example, enough salts or glucose to make the solution isotonic with blood. The aqueous solutions should be suitably buffered (preferably to a pH of from 3 to 9), if necessary. The preparation of suitable parenteral formulations under sterile conditions is readily accomplished by standard pharmaceutical techniques well-known to those skilled in the art.

Sterile injectable solutions can be prepared by mixing the repairing composition with an appropriate solvent and one or more of the aforementioned carriers, followed by sterile filtering. In the case of sterile powders suitable for use in the preparation of sterile injectable solutions, preparation methods may include drying in vacuum and lyophilization.

As used herein, the term “about” when combined with a value refers to plus and minus 10% of the reference value. For example, a length of about 1000 nanometers (nm) refers to a length of 1000 nm+−100 nm.

It is noted that as used herein and in the appended claims, the singular forms “a,” “an,” and “the” include plural referents unless the context clearly dictates otherwise. Thus, for example, reference to “a polynucleotide” includes a plurality of such polynucleotides and reference to “the polypeptide” includes reference to one or more polypeptides and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

In those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

Various embodiments and aspects of the present invention as delineated hereinabove and as claimed in the claims section below find experimental support in the following examples.

### EXAMPLES

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Strategies for Protein Purification and Characterization—A Laboratory Course Manual” CSHL Press (1996); all of which are incorporated by reference. Other general references are provided throughout this document.

Materials and Methods

The generation of chemotherapy-educated MSCs or MSC-derived conditioned medium: Murine MSCs were isolated from bone marrow aspirates and cultured in culture dishes in minimum essential medium-alpha (αMEM) supplemented with 10% FBS 1% L-glutamine, 1% sodium-pyruvate, and 1% streptomycin were used. The purification of MSCs was performed based on their adhesion abilities to plastic culture dishes. Medium was changed every 3 days until the hematopoietic cells were washed away, leaving the adhered MSC homogenous culture. The achieved murine MSCs were expanded and passaged, while cells up to passage 10 were used for the experiments.

To generate chemotherapy-activated MSCs, cultured MSCs were exposed to paclitaxel (100 nM), cisplatin (3 μM), gemcitabine (10 nM), or vehicle control for 24 hours. To generate MSC-derived conditioned medium (CM), the chemotherapy-educated MSCs (as above) were re-seeded in serum-free medium at a concentration of 1×106 cells/ml. After 72 hours, CM was collected.

Animal models, treatments, and live imaging: The use of animals and experimental protocols were approved by the Animal Care and Use Committee of the Technion (Envigo, Israel). Murine model of acute pulmonary inflammation was induced by a single intratracheal instillation of 5 mg/kg of lipopolysaccharides from Escherichia coli (Sigma Aldrich, Israel), as was previously reported. Mice were left either untreated or treated with anti-IL-6R antibody (10 mg/kg; BioXcell, NH, USA), CM of control non-activated cells, or with CM of activated cell therapy. The treatment was given once daily for 5 days. The efficacy of the route of delivery was determined, using intratracheal or intraperitoneal administration. Lung inflammation was evaluated by computer tomography (CT) screening using SKYSCAN 1276 micro-CT scanner (Bruker Corporation, MA, USA). After 5 days mice were anesthetized and sacrificed. Bronchoalveolar lavage (BAL) was performed by washing the lungs with Hank's balanced salt solution (HBSS) supplemented with 100 μM ethylenediaminetetraacetic acid (EDTA, both were from Biological industries ltd, Israel) and flow cytometry was used for the analysis of myeloid derived suppressor cells (using CD11b+ and Gr1+ surface markers). Lungs and additional organs were harvested for histopathological evaluation. For safety experiment, healthy mice were treated with the activated cell product for 8 consecutive days in the same conditions mentioned above. The mice underwent histopathological examination.

Bone marrow derived macrophage isolation and differentiation: Bone marrow derived macrophages (BMDMs) were isolated as previously described. Briefly, bone marrow cells were flushed from the bone marrow, and subsequently cultured at a concentration of 500,000 cells/ml in the presence of macrophage colony stimulating factor (MCSF, 10 ng/ml, PeproTech, Israel) for seven days to induce macrophage differentiation. After seven days, medium was aspirated and replaced with fresh medium. The resulting population represents M0 macrophages. Macrophage skewing was achieved by treating M0 macrophages with CM of control MSCs or MSCs activated with paclitaxel or cisplatin. M1 (CD11b+F4/80+, CD11c+, CD206−) and M2 (CD11b+F4/80+, CD11c−, CD206+) phenotypes were confirmed using flow cytometry.

Cytokine array: Levels of the inflammatory cytokines in plasma from mice with LPS-induced pulmonary inflammation or from mice treated with the CM of paclitaxel-activated MSC were measured using a proteome profiler mouse XL cytokine array (ARY028, R&D systems, MN) in accordance with the manufacturer's instruction. The signals corresponding to each factor in the array were quantified by densitometry analysis. The ratio between the expression levels of the various factors in plasma of sick mice and mice treated with CM of paclitaxel-activated MSCs was calculated.

Histology: Lungs from control or CM-treated mice were harvested at endpoint and subsequently were fixed with paraformaldehyde. Paraffin-embedded tissues were sectioned and stained with Hematoxylin and Eosin (H&E) solution.

Statistical analysis: Data are expressed as mean±standard deviation (SD). The statistical significance of differences was assessed by one-way ANOVA, followed by Tukey ad hoc statistical test using GraphPad Prism 5 software (La Jolla, Calif.). Student t-test was used in some experiments when comparing only two groups. Differences between all groups were compared with each other and were considered significant at p values below 0.05.

### Example 1: Conditioned Medium of Chemotherapy-Activated MSC Increases Healing of Acute Respiratory Inflammation

MSC therapy is currently being evaluated for the treatment of several pathologies, including pathologies requiring regenerative medicine. In recent years, the therapeutic activity of MSC therapy has been tested for its ability to suppress acute inflammation and cytokine storm. For example, recent studies evaluated the ability of MSCs to inhibit COVID-19 pathologies associated with acute pulmonary inflammation and cytokine storm. However, so far, such therapies have not shown any therapeutic benefit due, in part, to insufficient activity and high variability in the treatment outcome.

The following experiment was conducted using cell therapy products from MSCs stimulated by anti-cancer drugs that induce their regenerative, anti-inflammatory and reparative activities. To study the therapeutic effect of activated cell therapy on acute pulmonary inflammation, a lipopolysaccharides (LPS)-induced model of acute pulmonary inflammation was implemented. Specifically, 10-to-12-week-old C57Bl mice were instilled with a single dose of lipopolysaccharides (LPS; 5 mg/kg) from E. coli. Mice were randomly divided into groups and either left untreated or treated intratracheally once daily with conditioned media from MSCs activated with different chemotherapies. On the 4th day of the experiment, pulmonary damage and inflammation in lungs were assessed by CT scanning and a day after the mice were sacrificed, BALF was collected, and organs were processed for histological assessment.

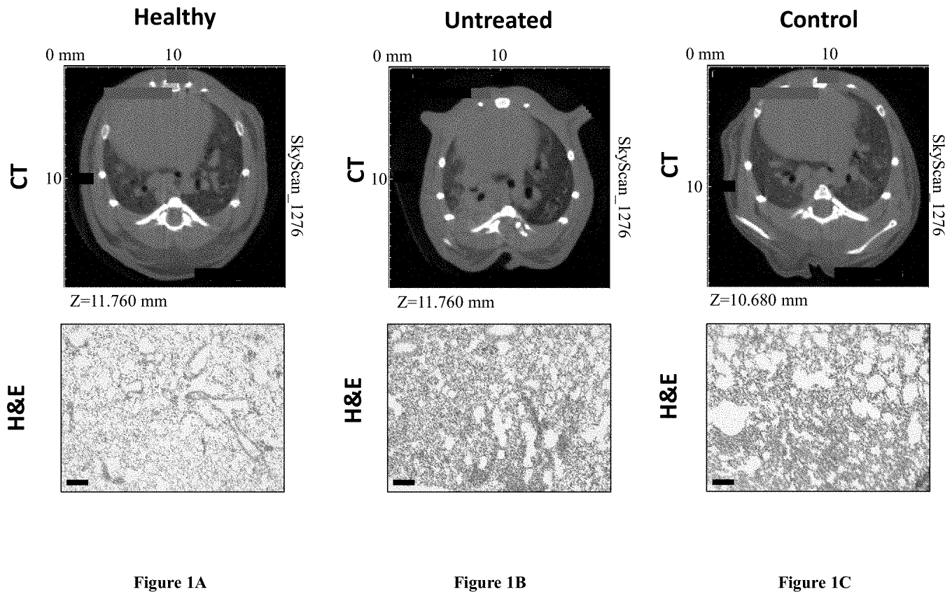

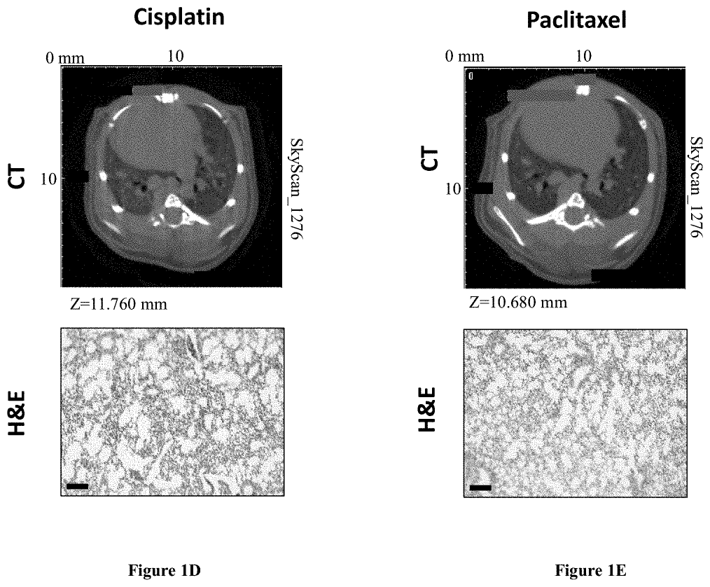

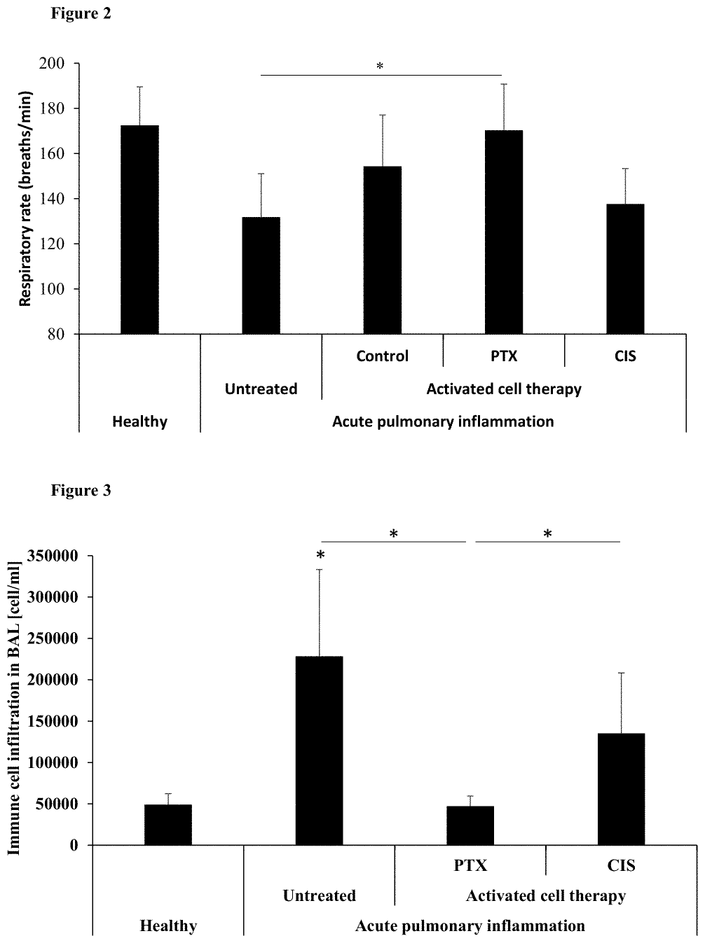

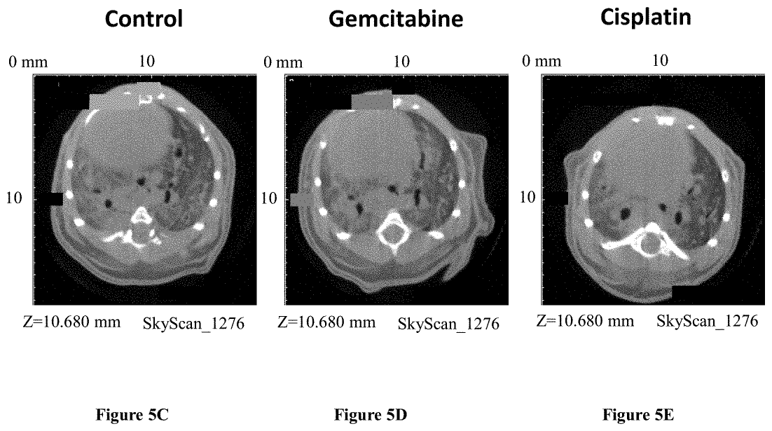

The effects of paclitaxel and cisplatin activated MSC products on respiratory inflammation burden were compared. To this end, CT scans and histology analysis of the lungs of sick or treated mice, demonstrated that intratracheal administration of the conditioned medium from paclitaxel-activated MSCs drastically reduced pulmonary inflammation and improved respiratory function when compared to the lungs of diseased mice or mice treated with the conditioned medium of non-activated, control MSCs (FIG. 1A-E). Cisplatin-activated MSC therapy also resulted in reduced pulmonary inflammation, however to a much lesser extent when compared to paclitaxel-activated therapy. Similar results were further supported by additional experiment in which the effect of paclitaxel-activated therapy was compared to the effect of gemcitabine- and cisplatin-therapies on mice with LPS-induced pulmonary inflammation. CT analysis showed that paclitaxel-activated MSC therapy was the most potent treatment as compared to gemcitabine and cisplatin (FIG. 5A-E). These data were supported by the analysis of breathing rate and function. Specifically, paclitaxel-activated therapy almost completely restored the respiratory rates of treated mice, which were reduced by severe pulmonary inflammation. Cisplatin-activated therapy resulted only in a partial improvement of the breathing rate of treated mice (FIG. 2).

To further study the severity of the inflammation in the lungs, BALF was collected and analyzed. As expected, acute pulmonary inflammation induced recruitment and homing of inflammatory cells into the lungs, as assessed by immune cell number in the BALF. However, BALF obtained from mice treated with paclitaxel-activated therapy showed a dramatic decrease in the number of infiltrating inflammatory cells (FIG. 3). Conditioned medium from cisplatin-activated cells resulted in a partial decrease in the cell infiltration in the lungs as well, but it was less effective than paclitaxel-activated therapy.

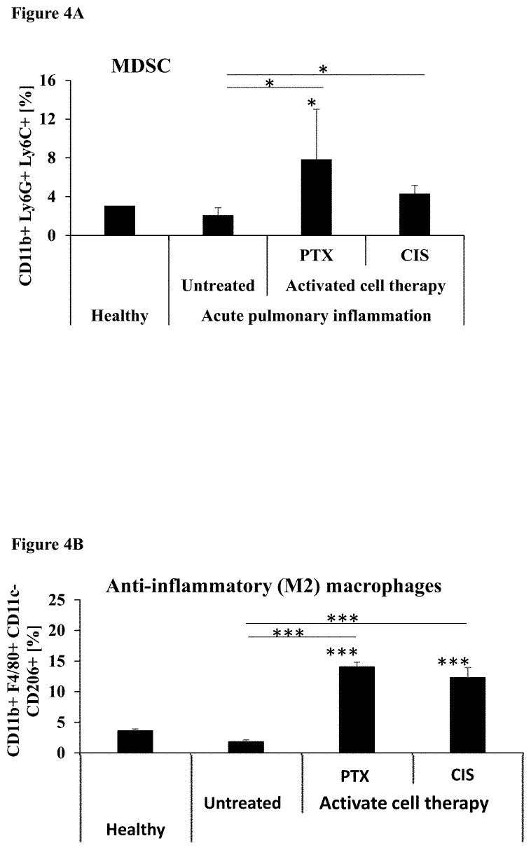

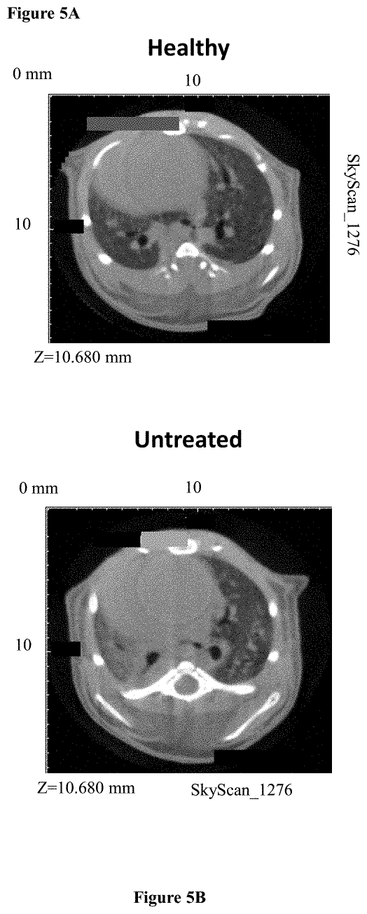

Next, flow cytometric analysis of cells in the BALF further confirmed that treatment with the conditioned medium of paclitaxel-activated MSCs improved the inflammatory condition in the lungs (FIG. 4A-B). Specifically, myeloid derived suppressor cells are increased in the lungs of mice treated with conditioned medium from paclitaxel-activated MSCs (FIG. 4A). This data is further supported by an observed shift in macrophages from naïve state to anti-inflammatory (M2) state when murine bone marrow was differentiated into macrophages in the presence of conditioned medium from paclitaxel-activated MSCs (FIG. 4B). Altogether these results demonstrate that conditioned medium of paclitaxel-activated MSCs induces immunosuppression and may be used as an effective therapy to treat acute pulmonary inflammation caused by infection.

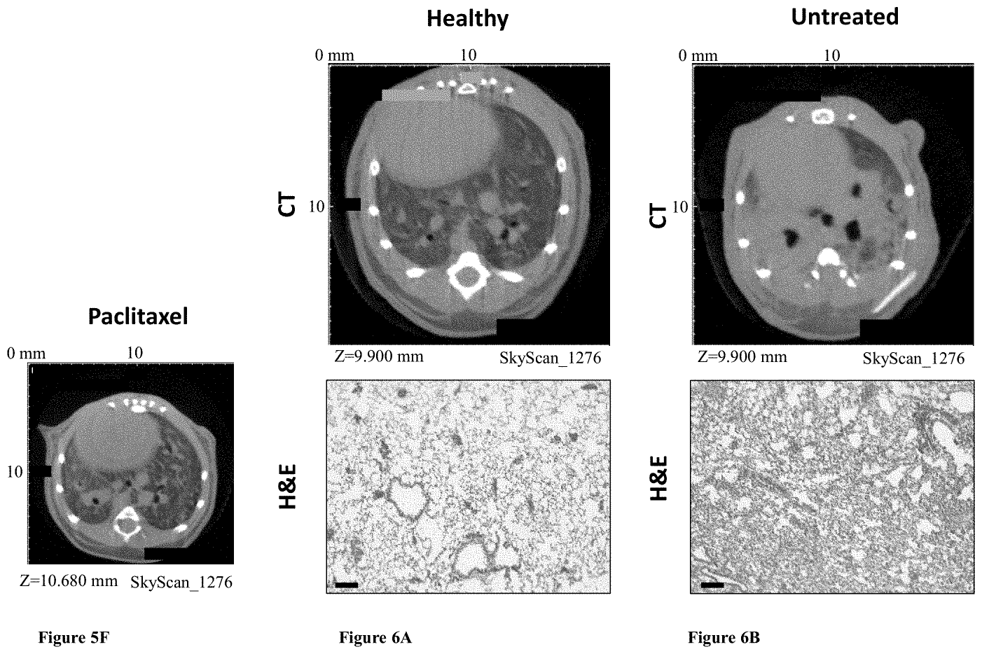

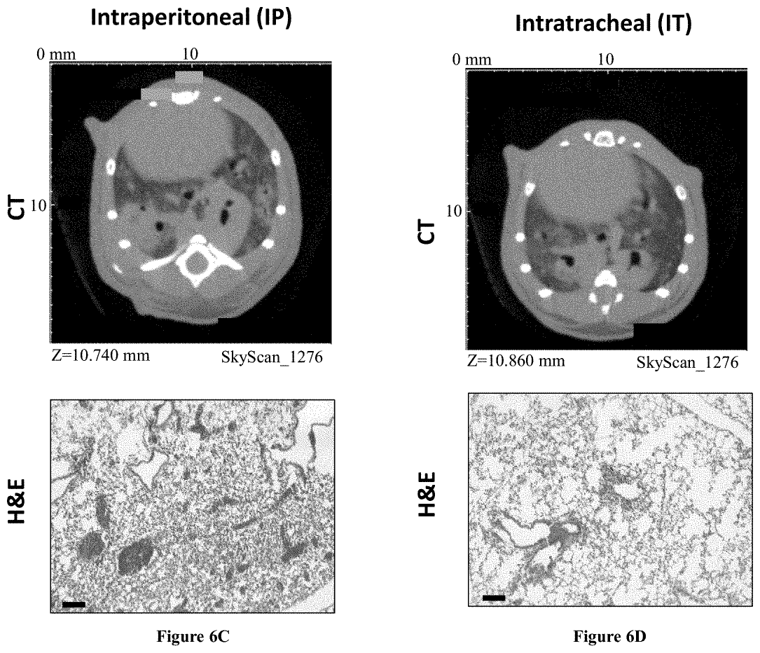

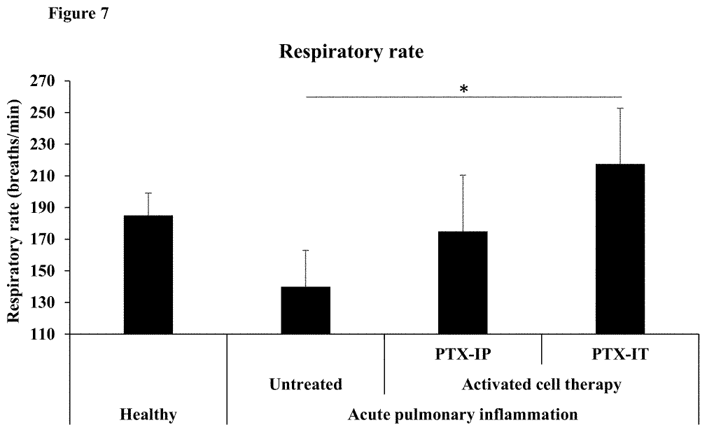

The route of treatment administration was next assessed. The conditioned medium from paclitaxel-activated MSCs was introduced once daily to mice with LPS-induced acute pulmonary inflammation, by either systemically administration via intraperitoneal injection, or locally via intratracheal instillation. Pulmonary inflammation severity was assessed by CT and histological evaluation (FIG. 6A-D). It was found that condition medium from paclitaxel activated MSCs improved the inflammatory state in the lungs and repaired their respiratory function, when compared to untreated diseased mice. Further, local instillation of the conditioned medium via the trachea resulted in a much better outcome, lower immune cell infiltration into the lungs and larger respiratory areas, when compared to systemic administration. Of note, the IP injection volume was almost 1.5 times higher than the volume introduced intratracheally. In addition, the healing properties of the activated cell therapy were confirmed by measuring respiratory rate of treated and control mice. Specifically, pulmonary inflammation caused a dramatic inhibition in respiratory function and inhibited the breathing rate of sick mice, while paclitaxel-activated cell therapy restored the breathing ability of treated mice (FIG. 7). Intratracheal instillation of conditioned medium obtained from paclitaxel-activated cells resulted in better recovery of respiratory function in diseased mice as compared to untreated control mice. Taken together these results suggest that paclitaxel-activated cell therapy is effective in treating acute inflammatory pulmonary conditions caused by infection.

### Example 2: Chemotherapy-Activated MSCs Suppress Cytokine Storm Syndrome

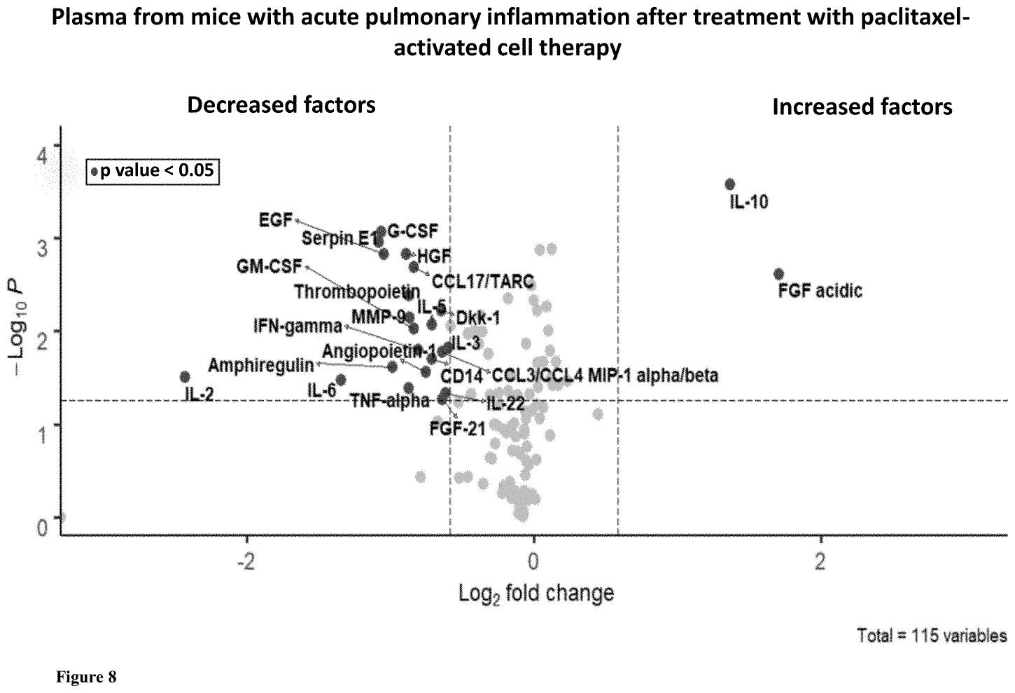

Cytokine storm syndrome (CSS) is another feature of the acute uncontrol immune activation followed by immune cell exhaustion. This is a life-threatening condition which results in multiple organ failure. COVID-19 infected patients may suffer from CSS, therefore there is an urgent need for treatments which suppress CSS or block its propagation. To assess whether conditioned medium from paclitaxel-activated MSC can suppress hypercytokinaemia and prevent tissue damage, the circulating levels of more than 100 inflammatory cytokines and chemokines were analyzed in mice with acute pulmonary inflammation that were treated with paclitaxel-activated MSC or in untreated control mice. The results demonstrated that acute respiratory inflammation induced massive secretion of pro-inflammatory mediators including IL-2, IL-3, IL-5, IL-6, IL22, CCL17, tissue necrosis factor alpha (TNFα), interferon-gamma (IFNγ), macrophage inflammatory protein-1-alpha (MIP-1α), granulocyte-colony stimulating factor (G-CSF), granulocyte-macrophage colony-stimulating factor (GM-CSF), and growth factors EGF, HGF, and FGF. CM treatment was given intratracheally, on a daily basis, for a 5-day period. At the endpoint, the therapeutic effect on cytokine storm and lung tissue condition was assessed. It was observed that treatment with the conditioned medium from paclitaxel-activated MSCs dramatically decreased plasma levels of all the above mentioned pro-inflammatory factors that had been overexpressed in sick mice (FIG. 8 and Table 1). In addition, the therapy significantly upregulated the circulating plasma levels of IL-10—a major immunosuppressive regulator, thus further inhibiting pulmonary inflammation. Overall, this therapy was found to be highly effective in controlling the cytokine storm induced by acute pulmonary inflammation and preventing lung tissue damage.

TABLE 1
Expression levels of cytokines in plasma of mice with
acute lung inflammation treated with paclitaxel-activated
cell therapy as compared to untreated mice.

| Protein | Direction of change | Fold change |
|---|---|---|
| FGF acidic | Increase | 3.25969 |
| IL-10 | Increase | 2.57995 |
| Amphiregulin | Decrease | 1.98049 |
| Angiopoietin-1 | Decrease | 1.68694 |
| MIP-1 alpha/beta | Decrease | 1.55926 |
| CCL17/TARC | Decrease | 1.78937 |
| CD14 | Decrease | 1.63778 |
| Dkk-1 | Decrease | 1.56609 |
| EGF | Decrease | 2.06175 |
| FGF-21 | Decrease | 1.55968 |
| G-CSF | Decrease | 2.09086 |
| GM-CSF | Decrease | 1.78551 |
| HGF | Decrease | 1.85267 |
| IFN-gamma | Decrease | 1.75029 |
| IL-2 | Decrease | 5.39049 |
| IL-3 | Decrease | 1.51572 |
| IL-5 | Decrease | 1.64018 |
| IL-6 | Decrease | 2.54251 |
| IL-22 | Decrease | 1.5302 |
| MMP-9 | Decrease | 1.82502 |
| Serpin E1 | Decrease | 2.12052 |
| Thrombopoietin | Decrease | 1.82951 |
| TNF-alpha | Decrease | 1.83284 |

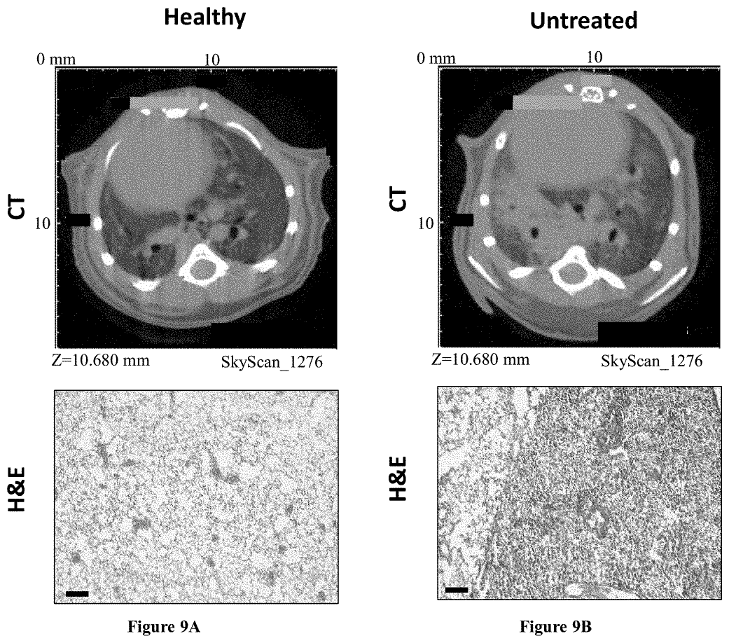

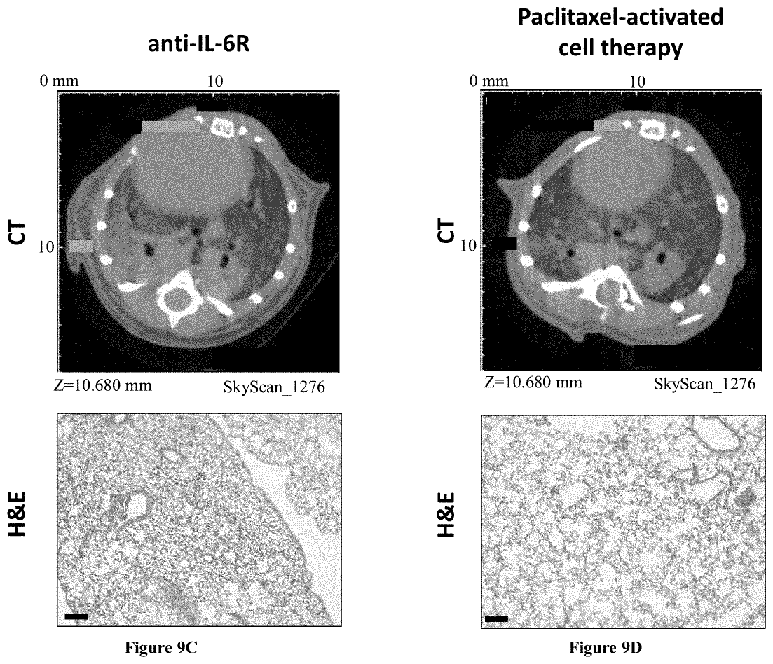

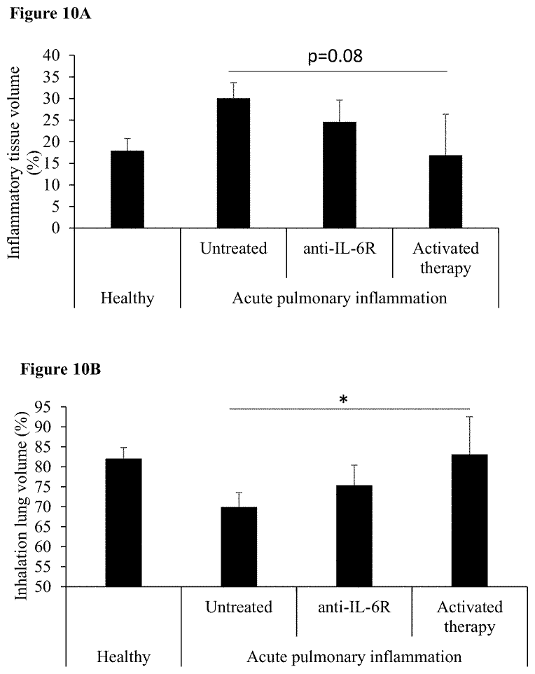

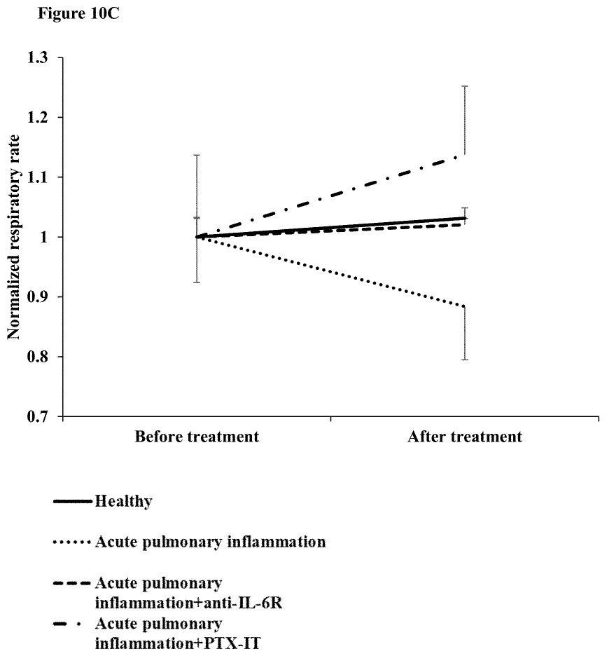

Siltuximab (anti-IL-6), Sarilumab and Tocilizumab (anti-IL-6R) are monoclonal antibodies with prominent immunosuppressive effects, which were approved for treatment of various inflammatory conditions including autoimmune diseases, rheumatoid arthritis, lymphoproliferative disorders and different malignancies, such as multiple myeloma, renal and prostate carcinomas. Moreover, Tocilizumab was approved for treatment of cytokine release syndrome after CAR-T therapy. Anti-IL-6R therapy is currently under FDA consideration for the treatment of COVID-19-induced pulmonary inflammation and cytokine storm. Recent clinical studies from Italy and China revealed rapid improvement of ARDS and CSS in patients, treated with anti-Il-6R therapy. Based on these data, the effect of chemotherapy-activated MSCs on CSS in mice having acute respiratory inflammation was compared to anti-IL-6R therapy. CT and histological staining demonstrated that anti-Il-6R therapy was effective in suppressing acute pulmonary inflammation (FIG. 9A-C). Anti-IL-6R injected intraperitoneally reduced inflammation (FIG. 10A), increased respiratory function (FIG. 10B-C), and inhibited inflammatory cell infiltration in the lungs (data not shown). However, CT scans also revealed diffused peripheral opacities in the lungs, immune cell infiltrate was still detected in the alveoli according to H&E staining, and the respiratory volume did not completely recover to healthy levels. In comparison, conditioned medium from paclitaxel-activated MSCs produced a greater recovery of the lungs and prevented extensive pulmonary damage. CT scans revealed no peripheral opacities, and only a small inflammatory area concentrated near the major bronchi (FIG. 9D, 10A). Minimal immune cell infiltrate, comparable to healthy lungs, was detected using histology analysis of the lung. Respiratory rate of treated mice was significantly improved as well. Furthermore, treatment with chemotherapy-activated cell products, resulted in improved pulmonary function, when compared to anti-Il-6R therapy. The respiratory volumes were restored to that found in healthy mice (FIG. 10B), and the breathing rate was substantially increased when compared to untreated diseased mice or diseased mice treated with anti-IL6R antibody (FIG. 10C). Taken together, CM from chemotherapy-activated MSCs was found to be superior in treating acute pulmonary inflammation as comparing to anti-IL-6R therapy. Without being bound to any one theory, this may be due to the reparative and immunosuppressive properties of chemo-activated MSC and their ability to suppress IL-6 and increase IL-10 levels, thus inhibiting pulmonary inflammation.

### Example 3: Chemotherapy-Activated MSC Product is Non-Toxic

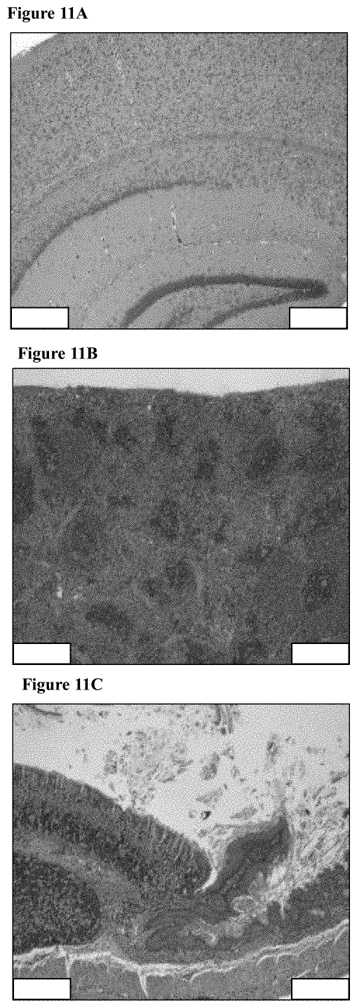

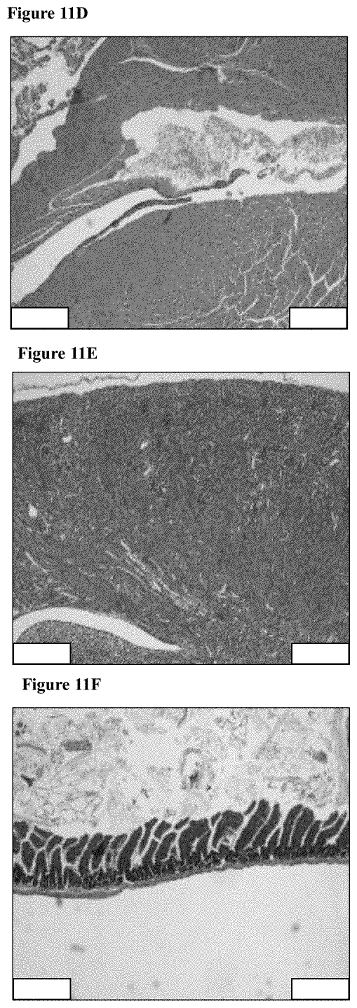

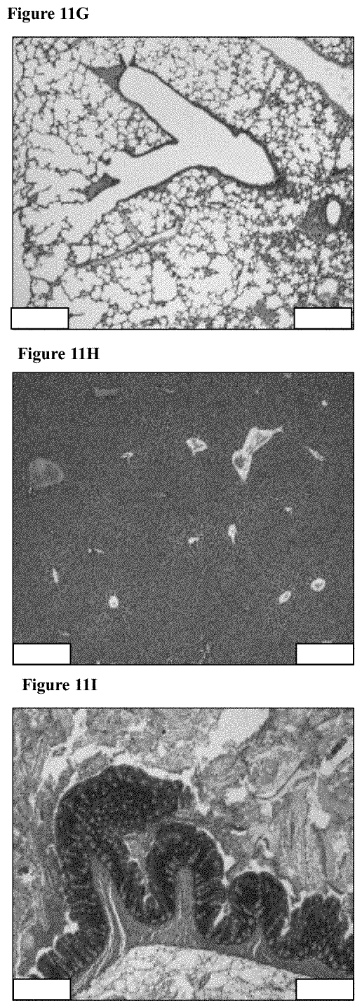

The safety profile associated with the administration of CM from paclitaxel-activated MSCs was assessed. To test this, a toxicity study was performed. For this purpose, naïve C57Bl mice were treated for 8 consecutive days (twice longer than usual experimental course) with conditioned medium from paclitaxel-activated MSCs. Treatment was either systemic, via intraperitoneal injection, or locally to the lungs, via intratracheal instillation, as described in the Methods section. At the endpoint, mice were euthanized and organs including brain (FIG. 11A), heart (FIG. 11D), lungs (FIG. 11G), liver (FIG. 11H), stomach (FIG. 11C), spleen (FIG. 11B), kidneys (FIG. 11E), small intestine (FIG. 11F), and colon (FIG. 11I) were fixated and underwent external histopathological evaluation. Unrelated to the route of delivery, paclitaxel-activated therapy, did not result in any pathological abnormalities and all the examined organs and tissues were found to be in a normal state (FIG. 11A-I). These results therefore provide further evidence that conditioned medium from paclitaxel-activated MSCs is safe and non-toxic.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1. A method of treating, ameliorating or preventing a cytokine storm in a subject in need thereof, the method comprising administering to said subject a pharmaceutical composition comprising an effective amount of MSCs contacted with a chemotherapy or conditioned medium (CM) derived from said MSCs contacted with said chemotherapy, thereby treating, ameliorating or preventing a cytokine storm in a subject.

2. A method of treating, ameliorating or preventing an acute respiratory disease or condition in a subject in need thereof, the method comprising administering to said subject a pharmaceutical composition comprising an effective amount of mesenchymal stem cells (MSCs) contacted with a chemotherapy or conditioned medium (CM) derived from said MSCs contacted with said chemotherapy, thereby treating, ameliorating or preventing an acute respiratory disease or condition in a subject.

3. The method of claim 2, wherein said acute respiratory disease or condition is acute respiratory distress syndrome (ARDS).

4. The method of claim 3, wherein said acute respiratory disease or condition is selected from severe lung trauma, sepsis, pulmonary infection, and acute pancreatitis.

5. The method of claim 1, wherein said subject suffers from a pulmonary infection.

6. The method of claim 5, wherein said pulmonary infection is selected from a bacterial infection, a viral infection and an infection consequent to aspiration of gastrointestinal contents.

7. The method of claim 6, wherein said viral infection is a corona virus infection, optionally wherein said corona virus is selected from, human coronavirus (HcoV)-NL63, HCoV-OC43, HCoV-229E, HCoV-HKUI, severe acute respiratory syndrome coronavirus (SARS-CoV-1), middle east respiratory syndrome coronavirus (MERS-CoV) and SARS-CoV-2.

8. (canceled)

9. The method of claim 2, wherein said acute respiratory disease or condition is an acute pulmonary infection and said method is a method for treating an acute pulmonary infection in a subject in need thereof.

10. The method of claim 2, wherein said acute respiratory disease or condition comprises at least one of: a cytokine storm, cytokine release syndrome, secondary hemophagocytic lymphohistiocytosis (sHLH), acute respiratory distress syndrome (ARDS), unremitting fever, cytopenia, or hyperferritinaemia.

11. The method of claim 1, wherein said subject does not suffer from lung fibrosis.

12. The method of claim 1, wherein said subject suffers from cytokine release syndrome.

13. The method of claim 1, comprising administering said conditioned media to said subject.

14. The method of claim 1, further comprising administrating to said subject an additional drug or therapy.

15. The method of claim 14, wherein the additional drug or therapy is selected from: an anti-cytokine therapy, an anti-viral drug, an analgesic drug, a corticosteroid and mechanical ventilation.

16. The method of claim 15, wherein said anti-viral drug is selected from remdesivir, ribavirin, oseltamivir, zanamivir and interferon-alpha 2b or said anti-cytokine therapy is selected from anti-IL-6, anti-IL6R and anti-IL1.

17. (canceled)

18. The method of claim 1, wherein said treating comprises reduction of a proinflammatory cytokine selected from IL-6, TNFα, IL-2, IL-3, G-CSF, MIP-1α, and INFγ in the blood of said subject, improvement in a respiratory parameter or a combination thereof.

19. (canceled)

20. (canceled)

21. The method of claim 1, wherein the composition is administered by oral, sublingual, subcutaneous, intramuscular, intravenous, topical, local, intratracheal, intranasal, transdermal or rectal administration.

22. (canceled)

23. The method of claim 1, wherein said chemotherapy is selected from paclitaxel, cisplatin and gemcitabine.

24. The method of claim 23, wherein said chemotherapy is selected from paclitaxel and cisplatin.

25. The method of claim 24, wherein said chemotherapy is paclitaxel.
